(19) United States
(12) Patent Application Publication
(10) Pub. No.: US 2019/0151351 A1
(43) Pub. Date: May 23, 2019

(54) REGULATORS OF ANAPLASTIC LYMPHOMA KINASE AND USES THEREOF

(71) Applicants: Celldex Therapeutics, Inc., Hampton, NJ (US); Yale University, New Haven, CT (US)

(72) Inventors: Joseph Schlessinger, Woodbridge, CT (US); Diego Alvarado, Madison, CT (US); Phillip B. Murray, Centennial, CO (US)

(73) Assignees: Celldex Therapeutics, Inc., Hampton, NJ (US); Yale University, New Haven, CT (US)

(21) Appl. No.: 16/150,421

(22) Filed: Oct. 3, 2018

Related U.S. Application Data
(62) Division of application No. 15/314,408, filed on Nov. 28, 2016, now abandoned.
(60) Provisional application No. 62/005,618, filed on May 30, 2014.

(51) Int. Cl. A61K 31/727; A61K 31/715; A61K 31/721; C07K 16/40; A61K 31/702; G01N 33/50; A61K 31/726; A61K 31/737; A61K 38/18; C12Q 1/48

(52) U.S. Cl. CPC A61K 31/727; G01N 2500/04; A61K 31/721; C07K 16/40; A61K 31/702; G01N 33/5058; G01N 33/5023; A61K 31/726; A61K 31/737; A61K 38/18; C12Q 1/485; G01N 2500/20; G01N 2333/91205; C07K 2317/75; C07K 2317/34; A61K 31/715

(57) ABSTRACT

Provided herein are ALK regulators for the treatment of diseases and disorders. For example, presented herein are ALK regulators, e.g., ALK activators, and methods of treatment and/or prevention of diseases, including neurodegenerative, neuromuscular and cognitive diseases or disorders, and methods of enhancing cognitive abilities using ALK regulators, e.g., ALK activators. Also provided herein are ALK regulators, e.g., ALK inhibitors, and methods of treatment and/or prevention of diseases such as hyperproliferative and neoplastic disorders associated with cells that express ALK, e.g., cells that exhibit increased or constitutive levels of ALK tyrosine phosphorylation using such ALK regulators, e.g., ALK inhibitors. Pharmaceutical compositions of said ALK regulators, e.g, ALK activators and inhibitors are likewise provided.

This application claims the benefit of priority of U.S. provisional application No. 62/005,618, filed on May 30, 2014, which is incorporated herein by reference in its entirety.

This application incorporates by reference a Sequence Listing submitted with this application as a text file entitled “12638-092-228_SEQ.txt” created on May 27, 2015 and having a size of 14,482 bytes.

### 1. INTRODUCTION

Provided herein are Anaplastic Lymphoma Kinase (ALK) regulators for the treatment of diseases and disorders. For example, presented herein are ALK regulators, e.g., ALK activators, and methods of treatment and/or prevention of diseases, including neurodegenerative, neuromuscular and cognitive diseases or disorders, and methods of enhancing cognitive abilities using ALK regulators, e.g., ALK activators. Also provided herein are ALK regulators, e.g., ALK inhibitors, and methods of treatment and/or prevention of diseases such as hyperproliferative and neoplastic disorders associated with cells that express ALK, e.g., cells that exhibit increased or constitutive levels of ALK tyrosine phosphorylation using such ALK regulators, e.g., ALK inhibitors. Pharmaceutical compositions of said ALK regulators, e.g, ALK activators and inhibitors are likewise provided.

### 2. BACKGROUND

Anaplastic Lymphoma Kinase (ALK) is a receptor tyrosine kinase (RTK) involved in neurogenesis during embryonic development. ALK is transiently expressed in specific regions of the central and peripheral nervous system, for example the mid-brain, thalamus, olfactory bulb, and peripheral ganglia. This expression is essential and is highest in the neonatal brain. Expression is maintained at low levels in the adult brain.

ALK has also been shown to be expressed in a number of disease states, such as cancer and other hyperproliferative and neoplastic disorders. Overexpression and/or constitutive activation (such as by fusion proteins) of ALK has been associated with oncogenic growth and the formation of tumors. Aberrant ALK signaling has been implicated in driving several types of cancer. Genomic translocations resulting in the fusion of the ALK kinase domain with the oligomerization region of intracellular proteins (e.g., EML4-ALK, NPM-ALK, etc.) have been identified in approximately 5% of non-small cell lung cancers, and approximately 60% of anaplastic large-cell lymphomas. Furthermore, ALK activation through overexpression, somatic mutation, or germ-line mutation occurs in approximately 15% of neuroblastoma cases, the most common extra-cranial tumor type in children. By analogy with other receptor tyrosine kinases (RTKs), upregulation of ALK signaling through ligand-driven mechanisms might also constitute a relevant driver of tumorigenesis.

The molecular events that lead to ALK activation in mammals remain elusive. While physiologically relevant ALK ligands have been firmly established in invertebrates (Jeb in Drosophila and HEN-1 in C. elegans), mammalian orthologs of these proteins have not been identified. The secreted proteins Pleiotrophin (PTN) and Midkine (MK), have been proposed as physiological ALK ligands, however recent work has thrown into question whether PTN and MK are bona fide ALK ligands.

The extracellular domain (ECD) of mammalian ALK includes two Meprin/A5/protein tyrosine phosphatase Mu (MAM) domains that flank a low-density lipoprotein class A (LDLa) domain, followed by a glycine-rich region and a potential EGF-like domain. The ECD is unique among RTKs, sharing only high sequence similarity in the glycine-rich region and EGF domain with LTK, also an orphan RTK. In addition, there is a highly basic >250 amino acid region at the N-terminus of ALK with no known function and without significant sequence identity to any other polypeptide.

### 3. SUMMARY

The ALK activators and inhibitors and uses described herein are based, in part, on the analyses presented herein that advance the elucidation of a physiological ligand for mammalian ALK by demonstrating the binding and activation of ALK via negatively charged carbohydrate-containing molecules, including heparin and other molecules. These analyses not only revealed that such molecules bind to and activate ALK, but also elucidated structural features of the molecules that are important for this binding and activation. Briefly, without wishing to be bound by any particular theory, the data presented herein demonstrate that such molecules are capable of binding to the ALK extracellular domain, e.g., the positively-charged N-terminal domain of the ALK extracellular domain, leading to the oligomerization, e.g., dimerization, and autophosphorylation of ALK, as well as the phosphorylation of downstream targets of ALK.

In certain embodiments, provided herein is a method of treating or preventing a neurodegenerative, neuromuscular or cognitive disease or disorder in a subject, comprising administering to a subject in need thereof a therapeutically effective amount of an anaplastic lymphoma kinase (ALK) regulator. Further provided herein is a method of treating or preventing a neurodegenerative, neuromuscular or cognitive disease or disorder in a subject, comprising administering to the subject in need thereof a therapeutically effective amount of an anaplastic lymphoma kinase (ALK) activator, wherein the ALK activator is capable of binding to the N-terminal domain of the ALK receptor. In certain specific embodiments, the regulator is an ALK activator. In certain specific embodiments, the ALK activator is capable of increasing ALK tyrosine phosphorylation above background ALK tyrosine phosphorylation in unstimulated neuroblastoma cells. In certain specific embodiments, the ALK activator is an antibody. In certain embodiments, the ALK activator binds to the N-terminal domain of ALK. In certain specific embodiments, the ALK activator is capable of binding to a heparin binding motif of the ALK receptor. In certain specific embodiments, the heparin binding motif of the ALK receptor is located in the region of amino acid 21 to amino acid 263 of human ALK. In certain specific embodiments, the ALK activator is capable of binding to a region within amino acid residues 48 to 65 of human ALK. In certain specific embodiments, the ALK activator is capable of binding to a region within amino acid residues 48 to 65 of SEQ ID NO:1. In certain specific embodiments, the ALK activator is capable of binding to the N-terminal domain of ALK with a K_{D} of less than or equal to 0.25 μM. In certain specific embodiments, the ALK activator is capable of binding to the heparin binding motif of the ALK receptor with a K_{D} of less than or equal to 0.25 μM. In certain specific embodiments, the binding of the ALK activator to the N-terminal domain of the ALK receptor, e.g., to the heparin binding motif of the ALK receptor, can be inhibited by sucrose-octasulfate (SOS) or by a monomeric sulfated glycosaminoglycan. In certain specific embodiments, the neurodegenerative disease or disorder is Alzheimer's disease. In certain specific embodiments, the ALK activator comprises a negatively charged carbohydrate. In certain specific embodiments, the ALK activator comprises a sulfated carbohydrate. In certain specific embodiments, the ALK activator comprises multiple ALK binding sites. In certain specific embodiments, the ALK activator is an oligosaccharide or polysaccharide. In certain specific embodiments, the oligosaccharide or polysaccharide has a chain length of at least 10, 15, 20, 25, 35, or at least 45 monosaccharides. In certain specific embodiments, the oligosaccharide or polysaccharide is negatively charged. In certain specific embodiments, the negatively charged oligosaccharide or polysaccharide has a charge density of at between 0.1 and 6 equivalents per mole of monosaccharide at a pH of 7.0. In certain specific embodiments, the oligosaccharide or polysaccharide comprises a sulfated oligosaccharide or polysaccharide. In certain specific embodiments, the oligosaccharide or polysaccharide has at least 2, 3, 4, 5, 6, or 7 sulfate groups per 10 monosaccharides. In certain specific embodiments, the ALK activator comprises a glycosaminoglycan. In certain specific embodiments, the glycosaminoglycan has at most 1, 2, 3, 4, 5, 6, 7, 8, 9, 10, 11, 12, 13, 14, 15, 16, 17, 18, 19, 20, 21, 22, 23, 24, or at most 25 N-Acetylated disaccharides per 100 disaccharide units. In certain specific embodiments, the glycosaminoglycan is substantially free of N-Acetylated disaccharides. In certain specific embodiments, the glycosaminoglycan has at least 50, 60, 70, 80, 90, or at least 100 N-Sulfate groups per 100 disaccharide units. In certain specific embodiments, the glycosaminoglycan has at least 50, 60, 70, 80, 90, 100, 110, 120, 130, 140, or at least 150 O-Sulfate groups per 100 disaccharide units. In certain specific embodiments, the glycosaminoglycan has at least 10, 20, 30, 40, 50, 60, 70, 80, or at least 90 trisulfated disaccharides per 100 disaccharide units. In certain specific embodiments, substantially all disaccharides of the glycosaminoglycan are trisulfated. In certain specific embodiments, the glycosaminoglycan is negatively charged. In certain specific embodiments, the glycosaminoglycan is a sulfated glycosaminoglycan. In certain specific embodiments, the glycosaminoglycan has at least 2, 3, 4, 5, 6, or 7 sulfate groups per 10 monosaccharides. In certain specific embodiments, the sulfated glycosaminoglycan is heparin. In certain specific embodiments, the glycosaminoglycan is oversulfated. In certain specific embodiments, the glycosaminoglycan has a chain length of at least 15 disaccharides. In certain specific embodiments, the glycosaminoglycan has a chain length of at least 20 disaccharides. In certain specific embodiments, the glycosaminoglycan has a chain length of at least 25 disaccharides. In certain specific embodiments, the ALK activator comprises dextran sulfate. In certain specific embodiments, the ALK activator comprises a proteoglycan. In certain specific embodiments, the ALK activator comprises a negatively charged proteoglycan. In certain specific embodiments, the negatively charged proteoglycan is a sulfated proteoglycan. In certain specific embodiments, the sulfated proteoglycan is a heparin proteoglycan. In certain specific embodiments, the sulfated proteoglycan is a chondroitin sulfate proteoglycan. In certain specific embodiments, the sulfated proteoglycan is a dermatan sulfate proteoglycan. In certain specific embodiments, the ALK activator associates with or binds to a growth factor, or the ALK activator facilitates the binding of a polypeptide to the ALK receptor. In certain specific embodiments, the polypeptide is a growth factor.

In certain embodiments, provided herein is a pharmaceutical composition suitable for intraventricular administration comprising an ALK activator. Further provided herein is a pharmaceutical composition suitable for intraventricular administration comprising an ALK activator, wherein the ALK activator binds to the N-terminal domain of the ALK receptor, for example, wherein the ALK activator is capable of binding to the heparin binding motif of the ALK receptor. In certain specific embodiments, 1.0 μM of the ALK activator is capable of increasing ALK tyrosine phosphorylation above background ALK tyrosine phosphorylation in unstimulated neuroblastoma cells. In certain specific embodiments, the ALK activator is an antibody. In certain specific embodiments, the ALK activator is capable of binding to the heparin binding motif of ALK. In particular embodiments, the ALK activator binds within amino acid residues 48 to 65 of human ALK, e.g., within amino acids 48 to 65 of SEQ ID NO:1. In certain specific embodiments, the ALK activator is capable of binding to the N-terminal domain of the ALK receptor with a K_{D} of less than or equal to 0.25 μM. In certain specific embodiments, the ALK activator is capable of binding to the heparin binding motif of the ALK receptor with a K_{D} of less than or equal to 0.25 μM. In certain specific embodiments, the binding of the ALK activator to the N terminal domain of ALK, for example, the heparin binding motif of the ALK receptor, can be inhibited by sucrose-octasulfate (SOS) or by a monomeric sulfated glycosaminoglycan. In certain specific embodiments, the ALK activator comprises a carbohydrate. In certain specific embodiments, the ALK activator comprises a negatively charged carbohydrate. In certain specific embodiments, the ALK activator comprises a sulfated carbohydrate. In certain specific embodiments, the ALK activator comprises multiple ALK binding sites. In certain specific embodiments, the ALK activator comprises an oligosaccharide or polysaccharide. In certain specific embodiments, the ALK activator comprises a negatively charged oligosaccharide or polysaccharide. In certain specific embodiments, the ALK activator comprises a sulfated oligosaccharide or polysaccharide. In certain specific embodiments, the oligosaccharide or polysaccharide has a chain length of at least 10, 15, 20, 25, 35, or at least 45 monosaccharides. In certain specific embodiments, the negatively charged oligosaccharide or polysaccharide has a charge density of at between 0.1 and 6 equivalents per mole of monosaccharide at a pH of 7.0. In certain specific embodiments, the oligosaccharide or polysaccharide has at least 2, 3, 4, 5, 6, or 7 sulfate groups per 10 monosaccharides. In certain embodiments, the ALK activator comprises a glycosaminoglycan. In certain specific embodiments, the glycosaminoglycan is negatively charged. In certain embodiments, the ALK activator comprises a sulfated glycosaminoglycan. In certain specific embodiments, the glycosaminoglycan has at most 1, 2, 3, 4, 5, 6, 7, 8, 9, 10, 11, 12, 13, 14, 15, 16, 17, 18, 19, 20, 21, 22, 23, 24, or at most 25 N-Acetylated disaccharides per 100 disaccharide units. In certain specific embodiments, the glycosaminoglycan is substantially free of N-Acetylated disaccharides. In certain specific embodiments, the glycosaminoglycan has at least 50, 60, 70, 80, 90, or at least 100 N-Sulfate groups per 100 disaccharide units. In certain specific embodiments, the glycosaminoglycan has at least 50, 60, 70, 80, 90, 100, 110, 120, 130, 140, or at least 150 O-Sulfate groups per 100 disaccharide units. In certain specific embodiments, the glycosaminoglycan has at least 10, 20, 30, 40, 50, 60, 70, 80, or at least 90 trisulfated disaccharides per 100 disaccharide units. In certain specific embodiments, substantially all disaccharides of the glycosaminoglycan are trisulfated. In certain specific embodiments, the glycosaminoglycan is negatively charged. In certain specific embodiments, the glycosaminoglycan is a sulfated glycosaminoglycan. In certain specific embodiments, the glycosaminoglycan has at least 2, 3, 4, 5, 6, or 7 sulfate groups per 10 monosaccharides. In certain specific embodiments, the glycosaminoglycan is heparin. In certain specific embodiments, the glycosaminoglycan is oversulfated. In certain specific embodiments, the glycosaminoglycan has a chain length of at least 15 disaccharides. In certain specific embodiments, the glycosaminoglycan has a chain length of at least 20 disaccharides. In certain specific embodiments, the glycosaminoglycan has a chain length of at least 25 disaccharides. In certain specific embodiments, the ALK activator comprises dextran sulfate. In certain specific embodiments, the ALK activator comprises a proteoglycan. In certain specific embodiments, the ALK activator comprises a negatively charged proteoglycan. In certain specific embodiments, the negatively charged proteoglycan is a sulfated proteoglycan. In certain specific embodiments, the sulfated proteoglycan is a heparin proteoglycan. In certain specific embodiments, the sulfated proteoglycan is a chondroitin sulfate proteoglycan. In certain specific embodiments, the sulfated proteoglycan is a dermatan sulfate proteoglycan. In certain specific embodiments, the ALK activator associates with or binds to a polypeptide. In certain specific embodiments, the polypeptide is a growth factor. In certain specific embodiments, the ALK activator is an oligosaccharide or polysaccharide. In certain specific embodiments, the oligosaccharide or polysaccharide has a chain length of at least 25 monosaccharides. In certain specific embodiments, the oligosaccharide or polysaccharide has a chain length of at least 35 monosaccharides. In certain specific embodiments, the oligosaccharide or polysaccharide has a chain length of at least 45 monosaccharides. In certain specific embodiments, the oligosaccharide or polysaccharide is negatively charged.

In certain embodiments, provided herein is a method of treating or preventing an ALK receptor-associated disorder in a subject, comprising administering to a subject in need thereof a therapeutically effective amount of an ALK inhibitor, wherein the ALK inhibitor inhibits binding of heparin or a heparin/chondroitin sulfated growth factor to the ALK receptor. In certain specific embodiments, the ALK inhibitor inhibits binding of heparin to the N-terminal domain of the ALK receptor. In certain specific embodiments, the ALK inhibitor inhibits binding of heparin to the heparin binding motif of the ALK receptor. In certain specific embodiments, the N-terminal domain containing heparin binding motif comprises amino acid residues 48 to 65 of human ALK. In certain specific embodiments, the heparin binding motif comprises amino acid residues 48 to 65 of SEQ ID NO: 1. In certain specific embodiments, the ALK inhibitor inhibits binding of heparin to the ALK receptor as measured via surface plasmon resonance. In certain specific embodiments, the ALK inhibitor inhibits binding of heparin to the ALK receptor with an IC50 of less than or equal to 1 μM, 0.5 μM, 100 nM, 50 nM, or less than 10 nM. In certain specific embodiments, the ALK inhibitor binds to the ALK receptor. In certain specific embodiments, the ALK inhibitor is an antibody that specifically binds the ALK receptor. In certain specific embodiments, the ALK inhibitor is a soluble protein comprising a heparin-binding motif. In certain specific embodiments, the soluble protein comprises a heparin-binding portion of an ALK N-terminal domain. In certain specific embodiments, the soluble protein comprises a heparin-binding portion of amino acids 48 to 65 of human ALK. In certain specific embodiments, the soluble protein comprises a heparin-binding portion of amino acids 48 to 65 of SEQ ID NO:1. In certain specific embodiments, the ALK receptor-associated disorder is a hyperproliferative disorder. In certain specific embodiments, the hyperproliferative disorder is cancer. In certain specific embodiments, the cancer is a lymphoma. In certain specific embodiments, the lymphoma is an anaplastic large-cell lymphoma. In certain specific embodiments, the cancer is a non-small cell lung cancer, inflammatory breast cancer, medulloblastoma, rhabdomyosarcoma, colorectal cancer, pancreatic cancer, myofibroblastic tumors, Ewing's sarcomas, head-and-neck cancer, neurofibromatosis, ovarian cancer, or glioblastoma. In even more specific embodiments, the cancer is a neuroblastoma.

In certain embodiments, provided herein is a method of screening for an ALK ligand, comprising i) contacting an ALK-expressing cell with a test agent and heparin, and ii) measuring the level of ALK tyrosine phosphorylation; wherein an increase in ALK tyrosine phosphorylation in the presence of the test agent and heparin in comparison with the level of ALK tyrosine phosphorylation in the absence of the test agent indicates that the test agent is an ALK ligand. In certain embodiments, the ALK-expressing cell is a neuroblastoma cell.

In certain embodiments, provided herein is a method of screening for an ALK activator, comprising i) contacting an ALK-expressing cell with a test agent and heparin, and ii) measuring the level of ERK 1/2 phosphorylation, wherein an increase in ERK 1/2 phosphorylation in the presence of the test agent and heparin in comparison with the level of ERK 1/2 phosphorylation in the absence of the test agent indicates that the test agent is an ALK activator. In certain embodiments, the ALK-expressing cell is a neuroblastoma cell.

In certain embodiments, provided herein is a method of screening for an ALK activator, comprising i) contacting an ALK-expressing cell with a test agent and heparin, and ii) measuring the level of STAT3 phosphorylation, wherein an increase in STAT3 phosphorylation in the presence of the test agent and heparin in comparison with the level of STAT3 phosphorylation in the absence of the test agent indicates that the test agent is an ALK ligand. In certain embodiments, the ALK-expressing cell is a neuroblastoma cell.

In certain embodiments, provided herein is a method of screening for an ALK ligand, comprising i) contacting an ALK-expressing cell with a test agent and heparin, and ii) measuring the level of STAT5 phosphorylation, wherein an increase in STAT5 phosphorylation in the presence of the test agent and heparin in comparison with the level of STAT5 phosphorylation in the absence of the test agent indicates that the test agent is an ALK ligand. In certain embodiments, the ALK-expressing cell is a neuroblastoma cell.

In certain embodiments, provided herein is a method of screening for an ALK ligand, comprising i) contacting a neuronal cell with a test agent and heparin, and ii) measuring the level of neurite outgrowth; wherein an increase in neurite outgrowth in the presence of the test agent and heparin in comparison with the level of neurite outgrowth in the absence of the test agent indicates that the test agent is an ALK ligand.

In certain embodiments, provided herein is a method of screening for an ALK ligand, comprising i) combining the test agent with heparin and ALK, and ii) measuring the level of dimerization of ALK; wherein an increase in dimerization of ALK in the presence of the test agent and heparin in comparison with the level of dimerization of ALK in the absence of the test agent indicates that the test agent is an ALK ligand.

In certain embodiments, provided herein is a method of screening for an ALK ligand, comprising i) combining the test agent with heparin and the N-terminal domain of ALK, and ii) measuring the level of dimerization of the N-terminal domain of ALK; wherein an increase in dimerization of the N-terminal domain of ALK in the presence of the test agent and heparin in comparison with the level of dimerization of the N-terminal domain of ALK in the absence of the test agent indicates that the test agent is an ALK ligand. In certain embodiments, the N-terminal domain of ALK comprises amino acid 48 to amino acid 65 of human ALK. In certain embodiments, the N-terminal domain of ALK comprises amino acid 21 to amino acid 263 of human ALK. In certain embodiments, the level of dimerization of the N-terminal domain of ALK is measured by size exclusion chromatography combined with multiangle laser light scattering (SEC-MALLS).

In certain embodiments, provided herein is a method of screening for an ALK ligand, comprising i) contacting the ALK with a test agent and heparin and ii) measuring the level of binding of the test agent to ALK, wherein binding of the test agent to ALK indicates that the test agent is an ALK ligand. In certain embodiments, binding of the test agent to ALK is not competitive with heparin binding. In certain embodiments, binding of the test agent to ALK is competitive with heparin binding.

In certain embodiments, provided herein is a method of screening for an ALK ligand, comprising i) contacting the N-terminal domain of ALK with a test agent and heparin and ii) measuring the level of binding of the test agent to the N-terminal domain of ALK, wherein binding of the test agent to the N-terminal domain of ALK indicates that the test agent is an ALK ligand. In certain embodiments, the N-terminal domain of ALK comprises amino acid 48 to amino acid 65 of human ALK. In certain embodiments, the N-terminal domain of ALK comprises amino acid 21 to amino acid 263 of human ALK. In certain embodiments, binding of the test agent to the N-terminal domain of ALK is not competitive with heparin binding. In certain embodiments, binding of the test agent to the N-terminal domain of ALK is competitive with heparin binding. In certain embodiments, binding to the N-terminal domain of ALK is measured by isothermal titration calorimetry (ITC). In certain embodiments, binding to the N-terminal domain of ALK is measured by surface plasmon resonance (SPR).

In certain embodiments, provided herein is a method of screening for an ALK ligand, comprising i) contacting ALK with a test agent and heparin, and ii) measuring the level of complex formation between ALK, heparin, and the test agent; wherein formation of a complex between ALK, heparin, and the test agent indicates that the test agent is an ALK ligand. In certain embodiments, complex formation is measured by surface plasmon resonance (SPR).

### 4. BRIEF DESCRIPTION OF THE FIGURES AND ABBREVIATIONS

### 4.1 Brief Description of the Figures

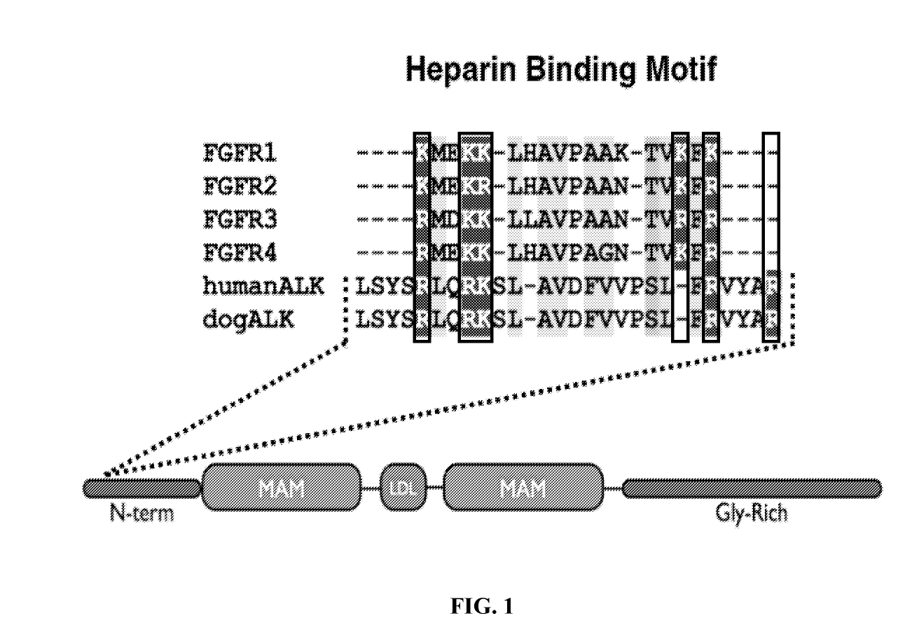

FIG. 1: A putative heparin-binding motif in the N-terminal domain of the ALK extracellular domain. The amino acid sequence of human ALK (a.a. 44-69 shown) and dog ALK (a.a. 44-69 shown) were aligned to the heparin-binding motif found in the FGF receptor (FGFR) family of RTKs. Positively charged amino acid residues (depicted with black backgrounds) are responsible for binding of FGFR to negatively charged heparin. These residues are largely conserved in ALK along with the intervening sequence, which constitutes the heparin-binding motif. The dotted lines correspond to the approximate location of the motif within the N-terminal domain of the ALK ECD (ALK ECD not drawn to scale).

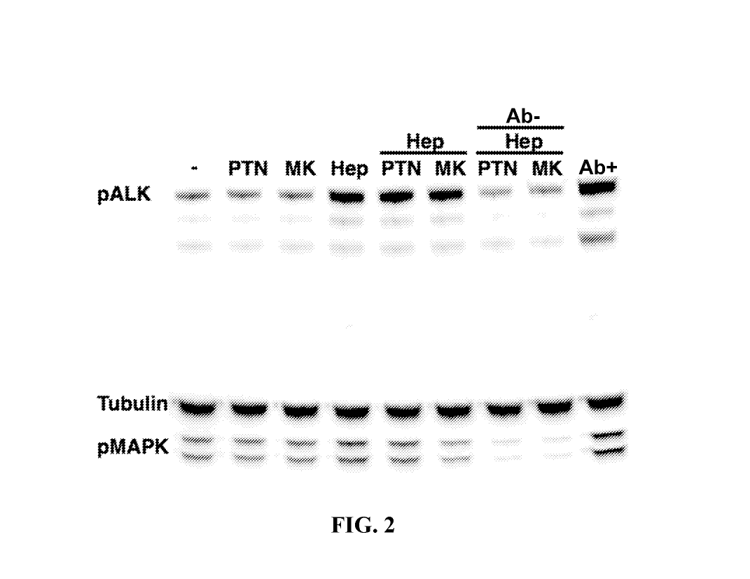

FIG. 2: Heparin, but neither PTN or MK, induces ALK tyrosine phosphorylation. Treating cells with 10 μg/mL of heparin stimulates ALK tyrosine phosphorylation and promotes MAPK activation in NB1 neuroblastoma cells. By contrast, neither PTN nor MK influence ALK phosphorylation. An agonistic anti-ALK monoclonal antibody was used as a positive control for activation of ALK and MAPK. An inhibitory anti-ALK antibody specifically blocked heparin-induced activation of ALK and MAPK. Without wishing to be bound by any particular mechanism or theory, this demonstrates that activation of ALK by heparin is mediated by direct interaction.

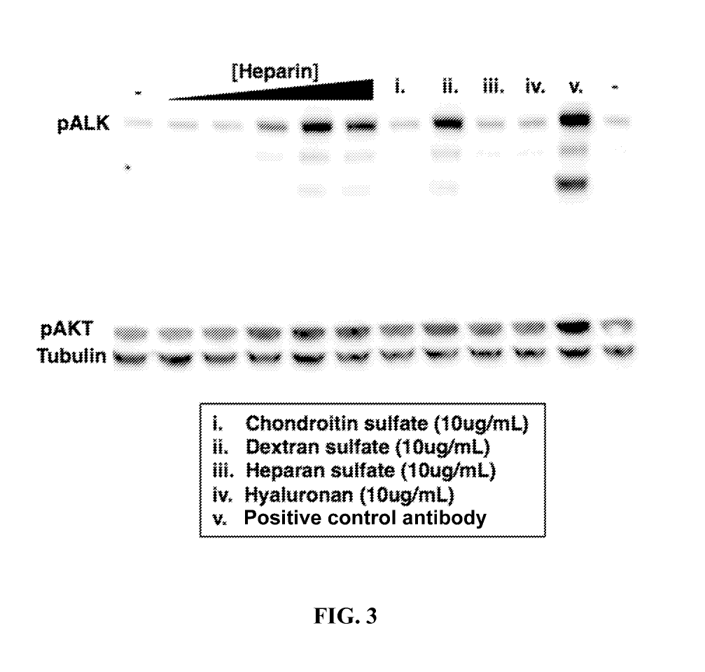

FIG. 3: Dose-dependent activation of ALK and AKT by heparin and effect of treatment with various other glycosaminoglycans.

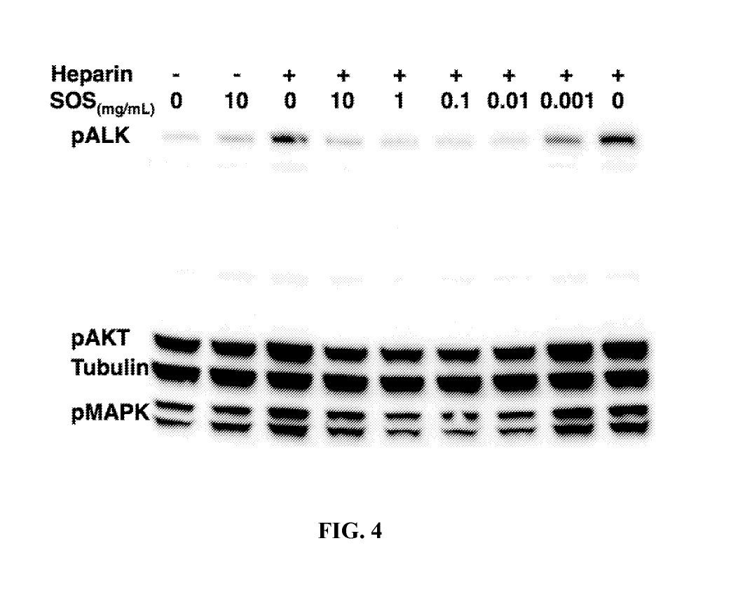

FIG. 4: Heparin induced activation of ALK is inhibited by sucrose-octasulfate (SOS). SOS is a heparin mimetic which contains the sugar and sulfate moieties to tightly bind heparin binding sites but lacks the length/size to induce dimerization (classically used to demonstrate the role of heparin in activating FGF receptors). Inhibition of heparin-induced pALK, pAKT and pMAPK by SOS is dose dependent.

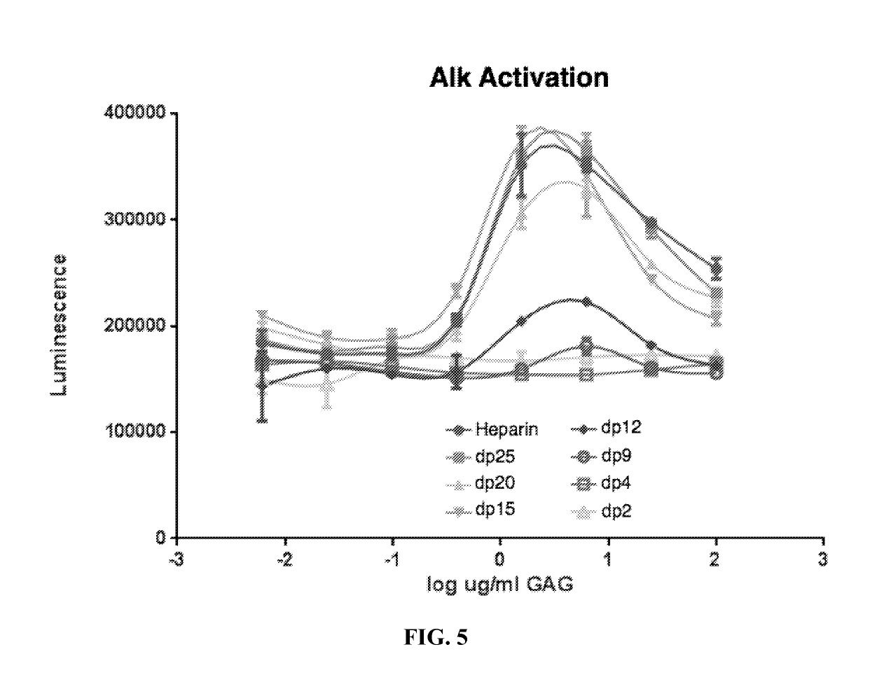

FIG. 5: ALK activation is influenced by heparin chain length. Heparin of 15 disaccharides (dp) or greater is able to induce ALK activation. Heparin of 12 dp also induces ALK activation, but to a lesser degree. NB1 cells were titrated with each heparin variant for 10 minutes, and ALK phosphorylation was read out by ELISA. The parabolic nature of ALK activation likely reflects monovalent saturation of ALK binding sites when heparin is in large excess.

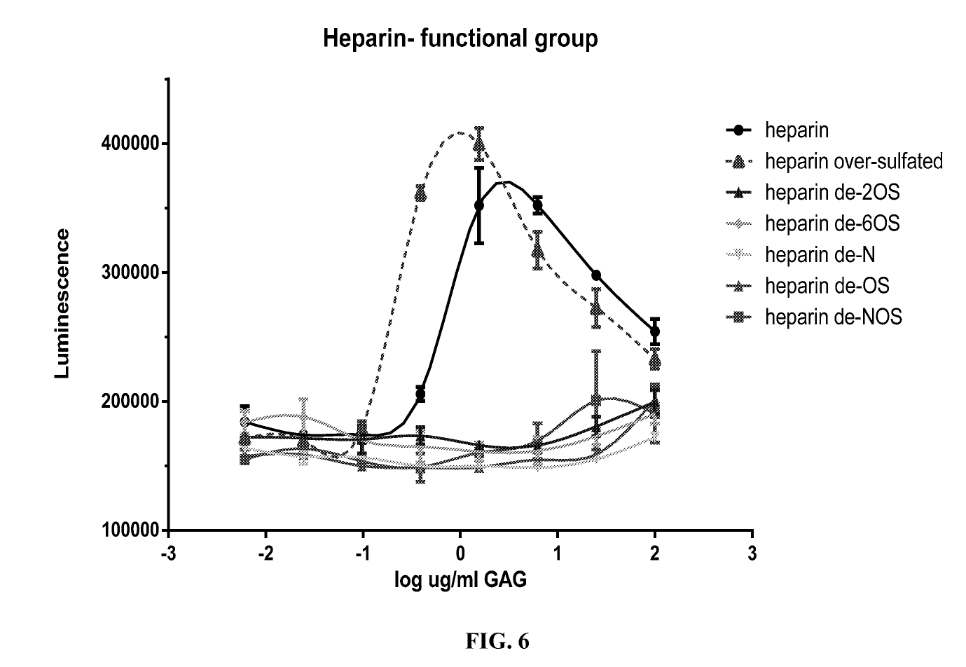

FIG. 6: ALK activation is dependent on heparin sulfation pattern. Removal of O-sulfated, or N-sulfated esters in the heparin chain results in a loss of ALK tyrosine phosphorylation as measured by ELISA. By contrast, heparin oversulfation (whereby most available hydroxyl groups are substituted by O-sulfate esters) promotes more robust ALK tyrosine phosphorylation.

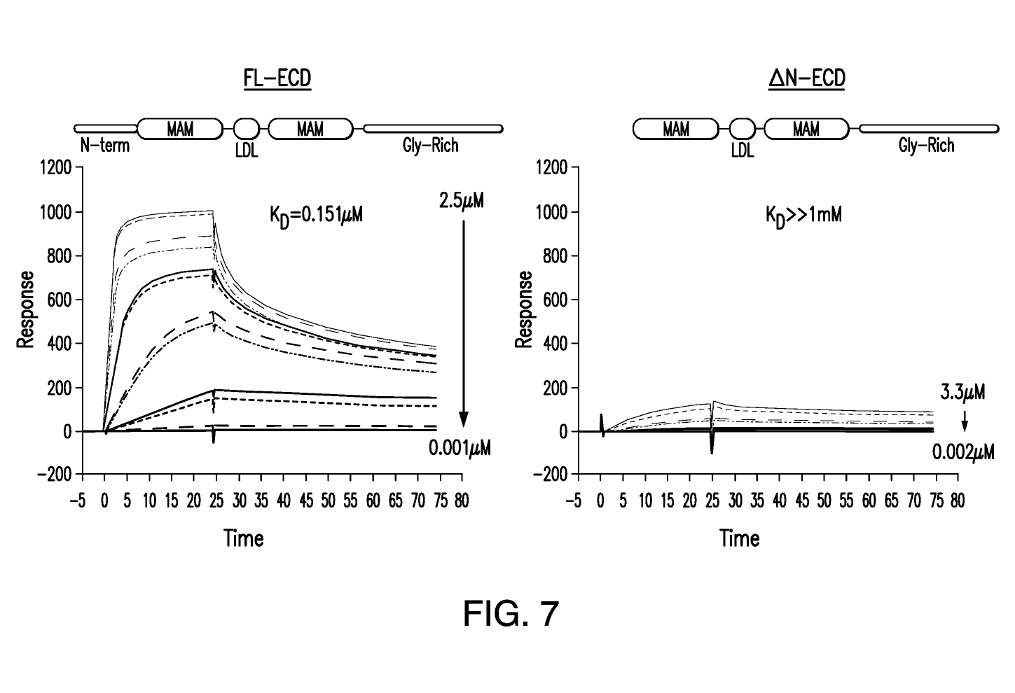

FIG. 7: SPR traces measuring the binding affinities of the ALK ECD and NTD deletion mutant to heparin. Using a BIACORE T100® instrument, a heparin surface was assembled on a BIACORE CM4® chip via amine coupling of NEUTRAVIDIN® to the chip and capturing biotinylated heparin (Sigma) on the surface. Heparin was captured at various concentrations in flow-cells 2, 3 and 4. Cell 1 was left unaltered and used as a reference cell to measure non-specific binding to the surface. The dog ALK ECD (FL-ECD) and a deletion mutant without the NTD (ΔN-ECD) were recombinantly expressed, purified, and flowed over the heparin surface. Using a steady-state model of binding affinity, the FL-ECD bound heparin with a K_{D} of 151 nM, whereas the ΔN-ECD mutant bound to heparin extremely weakly. The traces shown are reference subtracted with a background binding to the reference cell of <5%.

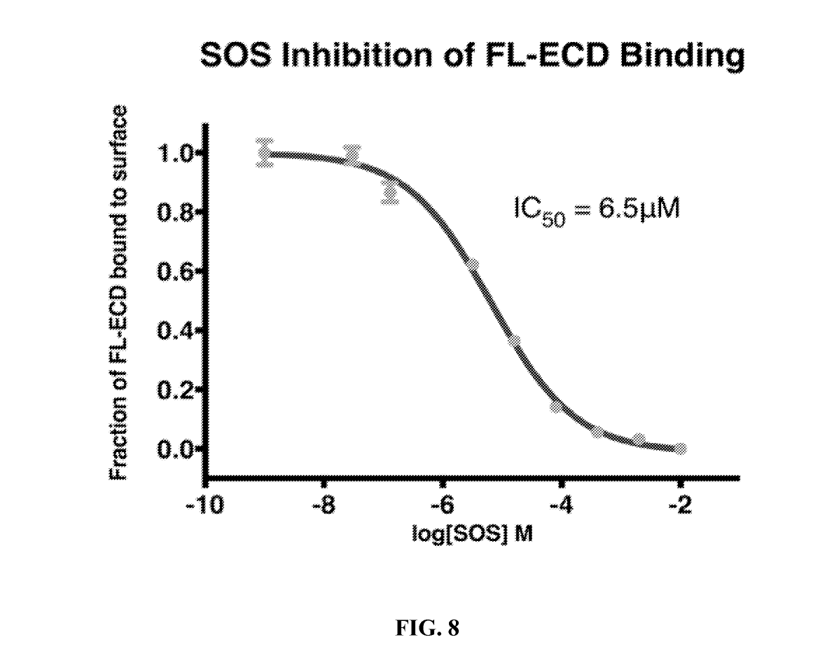

FIG. 8: Sucrose octasulfate (SOS) inhibits ALK binding to heparin in a dose-dependent manner, as measured by SPR. A titration of SOS was pre-incubated with 350 nM of FL-ECD. These complexes were then injected over the heparin surface. Rₘₐₓ values were taken for each concentration of SOS and an IC₅₀ was calculated to be 6.5 μM. The K_{D} for SOS/FL-ECD binding was calculated to be 2.25 μM using the Cheng-Prusoff equation.

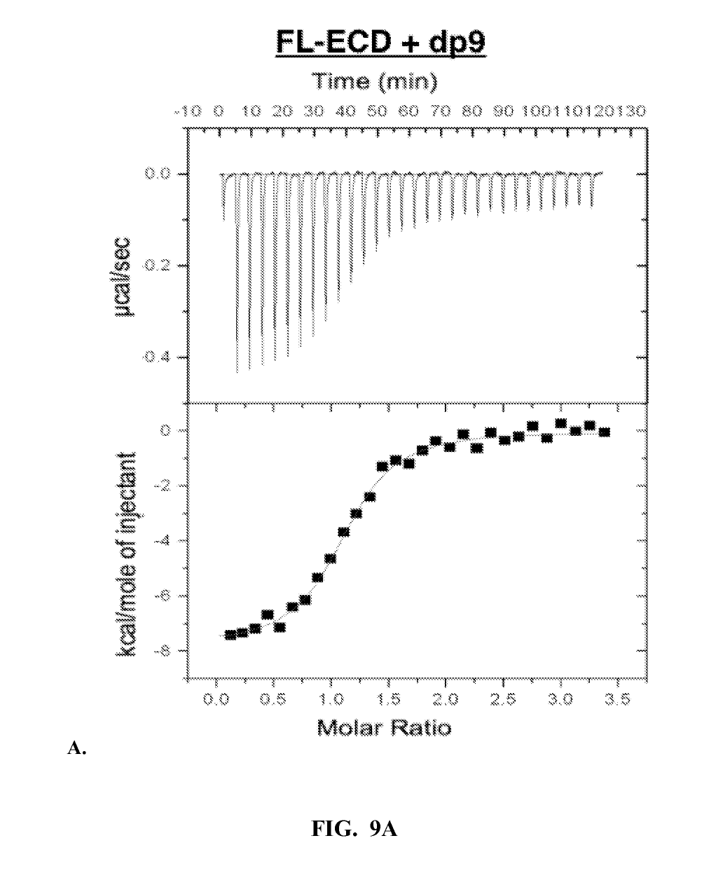

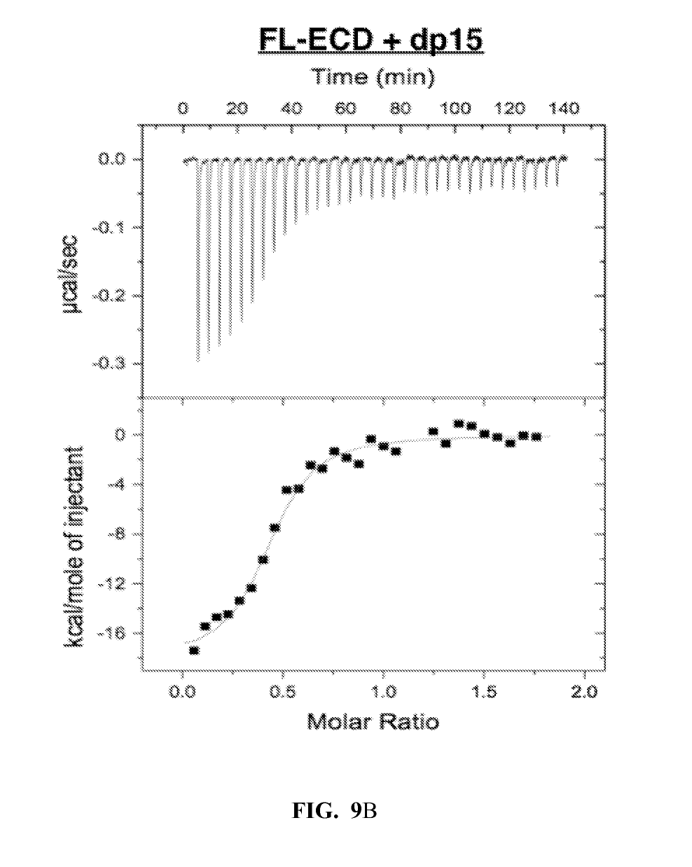

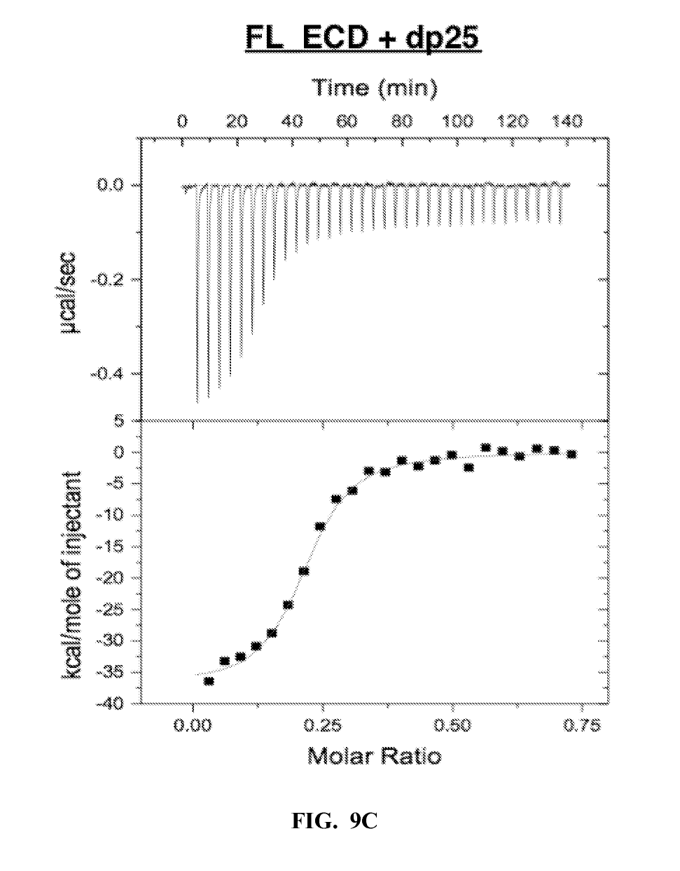

FIG. 9A-C: Stoichiometry and affinity of the ALK:Heparin complex as measured by ITC. ITC was performed by titrating heparins of specific lengths into a solution of FL-ECD. Data were collected, then processed and analyzed using Origin 5.0 with Microcal ITC software. Data were corrected for heat of dilution and then fitted to a one-site model by nonlinear least squares regression. A. 150 μM dp9 heparin was titrated into 10.0 μM FL-ALK. The K_{D} was 0.505 μM and the molar ratio was 1.0 heparin:0.91 ALK. B. 60 μM dp15 heparin was titrated into 6.0 μM FL-ALK. The K_{D} was 0.200 μM and the molar ratio was 1.0 heparin:2.3 ALK. C. 44 μM dp25 heparin was titrated into 8.3 μM FL-ALK. The K_{D} was 0.080 μM and the molar ratio was 1.0 heparin:4.7 ALK.

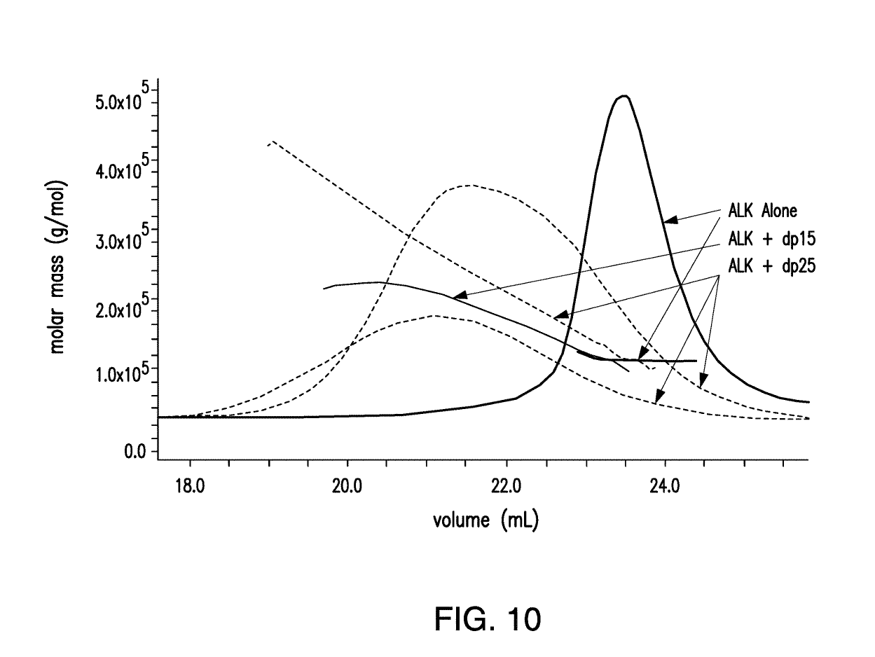

FIG. 10: Oligomerization of ALK on heparin as measured by SEC-MALLS. FL-ECD was mixed with dp15, dp25 or buffer and subjected to SEC-MALLS. FL-ECD+dp15 formed a FL-ECD dimer, FL-ALK+dp25 formed a FL-ECD tetramer/pentamer and FL-ECD+buffer formed a FL-ECD monomer. UV traces are thin lines, MALLS traces are thick lines.

### 4.2 ABBREVIATIONS AND CONVENTIONS

| | |
|---|---|
| ALK | Anaplastic lymphoma kinase |
| ALS | Amyotrophic lateral sclerosis |
| CSGAGS | Chondroitin/dermatan sulfate glycosaminoglycans |
| ECD | Extracellular domain |
| EGF | Epidermal growth factor |
| ELISA | Enzyme-linked immunosorbent assay |
| FBS | Fetal bovine serum |
| FGF | Fibroblastic growth factor |
| FGFR | Fibroblastic growth factor receptor |
| FL-ECD | Full-length extracellular domain (of dog ALK) |
| GAG | Glycosaminoglycan |
| Hep | Heparin |
| HSGAGS | Heparin/heparan sulfate glycosaminoglycans |
| ITC | Isothermal titration calorimetry |
| LDLa | Low-density lipoprotein class A |
| LTK | Leukocyte tyrosine kinase |
| MAM | Meprin/A5/protein tyrosine phosphatase Mu |
| MK | Midkine |
| NTD | N-terminal domain |
| PEG | Polyethylene glycol |
| PTN | Pleiotrophin |
| RTK | Receptor tyrosine kinase |
| SEC | Size exclusion chromatography |
| SEC-MALLS | Size-Exclusion Chromatography Combined with |
| | Multiangle Laser Light Scattering |
| SOS | Sucrose-octasulfate |
| SPR | Surface plasmon resonance |
| ΔN-ECD | Extracellular domain (of dog ALK) without the N- |
| | terminal domain |

### 5. DETAILED DESCRIPTION

In one aspect, described herein are regulators, e.g., activators, of ALK tyrosine kinase activity. With respect to ALK activators, such ALK activators can, for example, be used for the treatment and/or prevention of disorders such as cognitive disorders or neurodegenerative diseases e.g., Alzheimer's disease, Parkinson's disease and Amyotrophic Lateral Sclerosis. Accordingly, methods for the prevention and treatment of such disorders, e.g., neurodegenerative diseases or disorders, are also described herein.

In another aspect, described herein are regulators of ALK that are inhibitors of ALK. These ALK inhibitors can, for example, be used for the treatment and/or prevention of, for example, hyperproliferative disorders, such as cancers, including anaplastic large cell lymphoma, inflammatory myofibroblastic tumors, non-small cell lung cancer, diffuse large B-cell lymphoma, squamous cell carcinoma, and neuroblastoma, e.g., pediatric neuroblastoma. Accordingly, methods for the prevention and treatment of such disorders, for example hyperproliferative disorders, are also described herein.

As used herein, the terms “ALK” or “ALK receptor” or “ALK polypeptide” refer to mammalian ALK, e.g., human ALK, including, for example, native ALK, ALK isoforms, and ALK fusion polypeptides resulting from genomic rearrangements, e.g., translocations. Native ALK is a transmembrane polypeptide that comprises an extracellular domain (ECD), a transmembrane domain, and a cytoplasmic domain. The ALK ECD comprises a basic N-terminal domain, two Meprin/A5/protein tyrosine phosphatase Mu (MAM) domains that flank a low-density lipoprotein class A (LDLa) domain, and a membrane-proximal glycine-rich domain. In a specific embodiment, the ALK is a human ALK of the amino acid sequence of SEQ ID NO: 1. GenBank™ accession numbers NP_004295.2 provides an exemplary human ALK amino acid sequence. GenBank™ accession number NM_004304.4 provides an exemplary human ALK nucleic acid. Unless otherwise specified herein, references to particular amino acid residues of ALK correspond to the amino acid residues of the human form of ALK set forth in SEQ ID NO: 1 and reproduced below:

```
      1 MGAIGLLWLL PLLLSTAAVG SGMGTGQRAG SPAAGPPLQP REPLSYSRLQ RKSLAVDFVV
    61 PSLFRVYARD LLLPPSSSEL KAGRPEARGS LALDCAPLLR LLGPAPGVSW TAGSPAPAEA
  121 RTLSRVLKGG SVRKLRRAKQ LVLELGEEAI LEGCVGPPGE AAVGLLQFNL SELFSWWIRQ
  181 GEGRLRIRLM PEKKASEVGR EGRLSAAIRA SQPRLLFQIF GTGHSSLESP TNMPSPSPDY
  241 FTWNLTWIMK DSFPFLSHRS RYGLECSFDF PCELEYSPPL HDLRNQSWSW RRIPSEEASQ
  301 MDLLDGPGAE RSKEMPRGSF LLLNTSADSK HTILSPWMRS SSEHCTLAVS VHRHLQPSGR
  361 YIAQLLPHNE AAREILLMPT PGKHGWTVLQ GRIGRPDNPF RVALEYISSG NRSLSAVDFF
  421 ALKNCSEGTS PGSKMALQSS FTCWNGTVLQ LGQACDFHQD CAQGEDESQM CRKLPVGFYC
  481 NFEDGFCGWT QGTLSPHTPQ WQVRTLKDAR FQDHQDHALL LSTTDVPASE SATVTSATFP
  541 APIKSSPCEL RMSWLIRGVL RGNVSLVLVE NKTGKEQGRM VWHVAAYEGL SLWQWMVLPL
  601 LDVSDRFWLQ MVAWWGQGSR AIVAFDNISI SLDCYLTISG EDKILQNTAP KSRNLFERNP
  661 NKELKPGENS PRQTPIFDPT VHWLFTTCGA SGPHGPTQAQ CNNAYQNSNL SVEVGSEGPL
  721 KGIQIWKVPA TDTYSISGYG AAGGKGGKNT MMRSHGVSVL GIFNLEKDDM LYILVGQQGE
  781 DACPSTNQLI QKVCIGENNV IEEEIRVNRS VHEWAGGGGG GGGATYVFKM KDGVPVPLII
  841 AAGGGGRAYG AKTDTFHPER LENNSSVLGL NGNSGAAGGG GGWNDNTSLL WAGKSLQEGA
  901 TGGHSCPQAM KKWGWETRGG FGGGGGGCSS GGGGGGYIGG NAASNNDPEM DGEDGVSFIS
  961 PLGILYTPAL KVMEGHGEVN IKHYLNCSHC EVDECHMDPE SHKVICFCDH GTVLAEDGVS
1021 CIVSPTPEPH LPLSLILSVV TSALVAALVL AFSGIMIVYR RKHQELQAMQ MELQSPEYKL
1081 SKLRTSTIMT DYNPNYCFAG KTSSISDLKE VPRKNITLIR GLGHGAFGEV YEGQVSGMPN
1141 DPSPLQVAVK TLPEVCSEQD ELDFLMEALI ISKFNHQNIV RCIGVSLQSL PRFILLELMA
1201 GGDLKSFLRE TRPRPSQPSS LAMLDLLHVA RDIACGCQYL EENHFIHRDI AARNCLLTCP
1261 GPGRVAKIGD FGMARDIYRA SYYRKGGCAM LPVKWMPPEA FMEGIFTSKT DTWSFGVLLW
1321 EIFSLGYMPY PSKSNQEVLE FVTSGGRMDP PKNCPGPVYR IMTQCWQHQP EDRPNFAIIL
1381 ERIEYCTQDP DVINTALPIE YGPLVEEEEK VPVRPKDPEG VPPLLVSQQA KREEERSPAA
1441 PPPLPTTSSG KAAKKPTAAE ISVRVPRGPA VEGGHVNMAF SQSNPPSELH KVHGSRNKPT
1501 SLWNPTYGSW FTEKPTKKNN PIAKKEPHDR GNLGLEGSCT VPPNVATGRL PGASLLLEPS
1561 SLTANMKEVP LFRLRHFPCG NVNYGYQQQG LPLEAATAPG AGHYEDTILK SKNSMNQPGP
```

The ALK extracellular domain corresponds to amino acids 21-1038, the N-terminal domain corresponds to amino acids 21-263, the first MAM domain (MAM1) corresponds to amino acids 264-427, the LDLa domain corresponds to amino acids 437-473), the second MAM domain (MAM2) corresponds to amino acids 478-636, the glycine-rich domain corresponds to amino acids 816-940, the transmembrane domain corresponds to amino acids 1039-1059, the intracellular domain corresponds to amino acids 1060-1620, with the kinase domain corresponding to amino acids 1116-1392; the immature ALK sequence also includes a signal peptide at amino acids 1-20.

### 5.1 Activators of ALK

In one aspect, provided herein are regulators, e.g. activators, of ALK tyrosine kinase activity. In certain embodiments, the ALK activator is capable of increasing ALK tyrosine phosphorylation above background. In other embodiments, the ALK activator is capable of increasing neurite outgrowth above background. In certain embodiments, the ALK activator is capable of increasing ERK1/2 phosphorylation above background. In other embodiments, the ALK activator is capable of increasing AKT phosphorylation above background. In yet other embodiments, the ALK activator is capable of increasing STAT3 phosphorylation above background. In certain embodiments, the ALK activator is capable of increasing STAT5 phosphorylation above background. In other embodiments, the ALK activator is capable of increasing the dimerization or oligomerization of ALK or the ALK extracellular domain. In other embodiments, the affinity of the binding of a polypeptide for ALK is increased by the ALK activator. In more specific embodiments, such a polypeptide is a growth factor.

In one embodiment, the ALK activator increases ALK tyrosine phosphorylation above background in unstimulated neuroblastoma cells, that is, neuroblastoma cells in the absence of exogenous ALK modulators. For example, in one embodiment, the ALK activator increases ALK tyrosine phosphorylation above background in neuroblastoma cells cultured under normal cell culture conditions such as culturing in DMEM medium supplemented with 10% FBS and 1% penicillin/streptomycin. In certain embodiments, 0.01 to 10 μM of the ALK activator increases ALK tyrosine phosphorylation above background in unstimulated neuroblastoma cells. In a specific embodiment, 1 μM of the ALK activator increases ALK tyrosine phosphorylation above background in unstimulated neuroblastoma cells. In other specific embodiments, 0.01, 0.05, 0.1, 0.5, 5, or 10 μM of the ALK activator increases ALK tyrosine phosphorylation above background in unstimulated neuroblastoma cells. In certain embodiments, the ALK activator is present in a composition of ALK activator molecules with varying molecular weights. In certain, more specific embodiments, the average molecular weight of the ALK activator in such a composition is 5 kDa, 10 kDa, 15 kDa, 20 kDa, or 25 kDa. In other embodiments, the median molecular weight of the ALK activator is 5 kDa, 10 kDa, 15 kDa, 20 kDa, or 25 kDa. In certain embodiments, at least 50%, 60%, 70%, 80%, or at least 90% of the ALK activator molecules in such a composition are within a margin of at most 10%, at most 15%, at most 20%, or at most 25% below and above the average molecular weight. In certain embodiments, at least 50%, 60%, 70%, 80%, or at least 90% of the ALK activator molecules in such a composition are within a margin of at most 10%, at most 15%, at most 20%, or at most 25% below and above the median molecular weight. For example, if the average molecular weight is 15 kDa and the margin is 10%, then at least 50%, 60%, 70%, 80%, or at least 90% of the ALK activator molecules in such a composition have a molecular weight between 13.5 kDa and 16.5 kDa.

In certain embodiments, 0.01 to 10 μM of the ALK activator increases ALK tyrosine phosphorylation by 5%, 10%, 15%, 25%, 50%, or 100% in unstimulated neuroblastoma cells. In a specific embodiments, 1.0 μM of the ALK activator increases ALK tyrosine phosphorylation by 5%, 10%, 15%, 25%, 50%, or 100% in unstimulated neuroblastoma cells. In other specific certain embodiments, 0.01, 0.05, 0.1, 0.5, 5, or 10 μM of the ALK activator increases ALK tyrosine phosphorylation by 5%, 10%, 15%, 25%, 50%, or 100% in unstimulated neuroblastoma cells. In certain embodiments, the average molecular weight of the ALK activator is 5 kDa, 10 kDa, 15 kDa, 20 kDa, or 25 kDa. In other embodiments, the median molecular weight of the ALK activator is 5 kDa, 10 kDa, 15 kDa, 20 kDa, or 25 kDa.

In certain embodiments, 0.01 to 10 μM of the ALK activator increases ALK tyrosine phosphorylation 2, 3, 4, 5, 6, 7, 8, 9, or 10-fold in unstimulated neuroblastoma cells. In a specific embodiment, 1.0 μM of the ALK activator increases ALK tyrosine phosphorylation 2, 3, 4, 5, 6, 7, 8, 9, or 10-fold in unstimulated neuroblastoma cells. In other specific embodiments, 0.01, 0.05, 0.1, 0.5, 5, or 10 μM of the ALK activator increases ALK tyrosine phosphorylation 2, 3, 4, 5, 6, 7, 8, 9, or 10-fold in unstimulated neuroblastoma cells. In certain embodiments, the average molecular weight of the ALK activator is 5 kDa, 10 kDa, 15 kDa, 20 kDa, or 25 kDa. In other embodiments, the median molecular weight of the ALK activator is 5 kDa, 10 kDa, 15 kDa, 20 kDa, or 25 kDa.

In certain embodiments, the ALK activator is capable of increasing ALK tyrosine phosphorylation above background in any unstimulated ALK-expressing cell, e.g., an ALK-expressing cell or cell line, or a cell or cell line which has been transfected with an ALK vector to stably or transiently express ALK, known to one of skill in the art. In particular embodiments, 0.01 to 10 μM of the ALK activator is capable of increasing ALK tyrosine phosphorylation above background in an unstimulated ALK-expressing cell. In a specific embodiment, 1.0 μM of the ALK activator is capable of increasing ALK tyrosine phosphorylation above background in an unstimulated ALK-expressing cell. In other specific embodiments, 0.01, 0.05, 0.1, 0.5, 5, or 10 μM of the ALK activator is capable of increasing ALK tyrosine phosphorylation above background in an unstimulated ALK-expressing cell. In certain embodiments, 0.01 to 10 μM of the ALK activator increases ALK tyrosine phosphorylation by 5%, 10%, 15%, 25%, 50%, or 100% in an unstimulated ALK-expressing cell. In a specific embodiment, 1.0 μM of the ALK activator increases ALK tyrosine phosphorylation by 5%, 10%, 15%, 25%, 50%, or 100% in an unstimulated ALK-expressing cell. In other specific embodiments, 0.01, 0.05, 0.1, 0.5, 5, or 10 μM of the ALK activator increases ALK tyrosine phosphorylation by 5%, 10%, 15%, 25%, 50%, or 100% in an unstimulated ALK-expressing cell. In certain embodiments, 0.01 to 10 μM of the ALK activator increases ALK tyrosine phosphorylation by 2, 3, 4, 5, 6, 7, 8, 9, or 10-fold in an unstimulated ALK-expressing cell. In a specific embodiment, 1.0 μM of the ALK activator increases ALK tyrosine phosphorylation by 2, 3, 4, 5, 6, 7, 8, 9, or 10-fold in an unstimulated ALK-expressing cell. In other specific embodiments, 0.01, 0.05, 0.1, 0.5, 5, or 10 μM of the ALK activator increases ALK tyrosine phosphorylation by 2, 3, 4, 5, 6, 7, 8, 9, or 10-fold in an unstimulated ALK-expressing cell.

Tyrosine phosphorylation of ALK can be measured, for example, by an immunoblotting assay using an antibody that binds to phosphorylated ALK but not ALK that has not been phosphorylated (see, e.g., Moog-Lutz et al., 2005, J. Biol. Chem. 280(28):26039-26048). Phosphorylation of downstream targets of ALK can be measured, for example, by an immunoblotting assay using an antibody that binds to the phosphorylated target but not the target that has not been phosphorylated (see, e.g., Moog-Lutz et al., 2005). Any statistical method known to one of skill in the art can be used to confirm that the ALK tyrosine phosphorylation is significant and reproducible.

In certain embodiments, the ALK activator is capable of increasing neurite outgrowth above background in cells expressing ALK, e.g., PC 12 cells, stably or transiently expressing ALK. In particular embodiments, the ALK activator is capable of increasing neurite outgrowth above background in a neuronal cell line stably or transiently expressing ALK. Neurite outgrowth can be assayed, for example, as in Moog-Lutz et al., 2005.

In certain embodiments, 0.01 to 10 μM of the ALK activator is capable of increasing neurite outgrowth above background in a neuronal cell line stably or transiently expressing ALK. In a specific embodiment, 1.0 μM of the ALK activator is capable of increasing neurite outgrowth above background in a neuronal cell line stably or transiently expressing ALK. In other specific embodiments, 0.01, 0.05, 0.1, 0.5, 5, or 10 μM of the ALK activator is capable of increasing neurite outgrowth above background in a neuronal cell line stably or transiently expressing ALK.

In certain embodiments, 0.01 to 10 μM of the ALK activator is capable of increasing the percentage of cells extending neuritis by 5%, 10%, 15%, 25%, 50%, or 100% in a neuronal cell line stably or transiently expressing ALK. In a specific embodiment, 1.0 μM of the ALK activator is capable of increasing the percentage of cells extending neuritis by 5%, 10%, 15%, 25%, 50%, or 100% in a neuronal cell line stably or transiently expressing ALK. In other specific embodiments, 0.01, 0.05, 0.1, 0.5, 5, or 10 μM of the ALK activator is capable of increasing the percentage of cells extending neuritis by 5%, 10%, 15%, 25%, 50%, or 100% in a neuronal cell line stably or transiently expressing ALK.

In certain embodiments, 0.01 to 10 μM of the ALK activator is capable of increasing the percentage of cells extending neurites 2, 3, 4, 5, 6, 7, 8, 9, or 10-fold in a neuronal cell line stably or transiently expressing ALK. In a specific embodiment, 1.0 μM of the ALK activator is capable of increasing the percentage of cells extending neurites 2, 3, 4, 5, 6, 7, 8, 9, or 10-fold in a neuronal cell line stably or transiently expressing ALK. In other specific embodiments, 0.01, 0.05, 0.1, 0.5, 5, or 10 μM of the ALK activator is capable of increasing the percentage of cells extending neurites 2, 3, 4, 5, 6, 7, 8, 9, or 10-fold in a neuronal cell line stably or transiently expressing ALK.

In certain embodiments, the ALK activator is capable of increasing ERK1/2 phosphorylation above background in a cell line stably or transiently expressing ALK. In other embodiments, the ALK activator is capable of increasing AKT phosphorylation above background in a cell line stably or transiently expressing ALK. In other embodiments, the ALK activator is capable of increasing STAT1 phosphorylation above background in a cell line stably or transiently expressing ALK. In other embodiments, the ALK activator is capable of increasing STAT3 phosphorylation above background in a cell line stably or transiently expressing ALK. In other embodiments, the ALK activator is capable of increasing STAT5 phosphorylation above background in a cell line stably or transiently expressing ALK. In certain embodiments, 0.01 to 10 μM of the ALK activator is capable of increasing phosphorylation in one or more of ERK1/2, AKT, STAT1, STAT3, and STAT5. In a specific embodiment, 1.0 μM of the ALK activator is capable of increasing phosphorylation in one or more of ERK1/2, AKT, STAT1, STAT3, and STAT5. In other specific embodiments, 0.01, 0.05, 0.1, 0.5, 5, or 10 μM of the ALK activator is capable of increasing phosphorylation in one or more of ERK1/2, AKT, STAT1, STAT3, and STAT5. In certain embodiments, 0.01 to 10 μM of the ALK activator is capable of increasing phosphorylation in one or more of ERK1/2, AKT, STAT1, STAT3, and STAT5 by 5%, 10%, 15%, 25%, 50%, or 100% in a cell line stably or transiently expressing ALK. In a specific embodiment, 1.0 μM of the ALK activator is capable of increasing phosphorylation in one or more of ERK1/2, AKT, STAT1, STAT3, and STAT5 by 5%, 10%, 15%, 25%, 50%, or 100% in a cell line stably or transiently expressing ALK. In other specific embodiments, 0.01, 0.05, 0.1, 0.5, 5, or 10 μM of the ALK activator is capable of increasing phosphorylation in one or more of ERK1/2, AKT, STAT1, STAT3, and STAT5 by 5%, 10%, 15%, 25%, 50%, or 100% in a cell line stably or transiently expressing ALK. In certain embodiments, 0.01 to 10 μM of the ALK activator is capable of increasing phosphorylation 2, 3, 4, 5, 6, 7, 8, 9, or 10-fold in one or more of ERK1/2, AKT, STAT1, STAT3, and STAT5 in a cell line stably or transiently expressing ALK. In a specific embodiment, 1.0 μM of the ALK activator is capable of increasing phosphorylation 2, 3, 4, 5, 6, 7, 8, 9, or 10-fold in one or more of ERK1/2, AKT, STAT1, STAT3, and STAT5 in a cell line stably or transiently expressing ALK. In a specific embodiment, 0.01, 0.05, 0.1, 0.5, 5, or 10 μM of the ALK activator is capable of increasing phosphorylation 2, 3, 4, 5, 6, 7, 8, 9, or 10-fold in one or more of ERK1/2, AKT, STAT1, STAT3, and STAT5 in a cell line stably or transiently expressing ALK.

In certain embodiments, the ALK activator is capable of increasing the oligomerization of ALK or the ALK extracellular domain. In specific embodiments, the ALK activator is capable of increasing the oligomerization of ALK or the ALK extracellular domain as measured by SEC-MALLS. In other specific embodiments, the ALK activator is capable of increasing the dimerization of ALK or the ALK extracellular domain as measured by SEC-MALLS. In other specific embodiments, the ALK activator is capable of increasing the trimerization and tetramerization of the ALK extracellular domain as measured by SEC-MALLS. In other specific embodiments, the ALK activator is capable of increasing the pentamerization of the ALK extracellular domain as measured by SEC-MALLS. In specific embodiments, the ALK activator is capable of increasing the oligomerization of ALK or the ALK extracellular domain as measured by isothermal titration calorimetry. In more specific embodiments, the ALK activator is capable of increasing the dimerization of ALK or the ALK extracellular domain by 10, 20, 30, 40, 50, or 100% as measured by SEC-MALLS. In more specific embodiments, the ALK activator is capable of increasing the dimerization of ALK or the ALK extracellular domain by 10, 20, 30, 40, 50, or 100% as measured by isothermal titration calorimetry.

In certain embodiments, the activity of the ALK activator, as, for example, measured by one of the assays described above, is increased by binding of the ALK activator to a growth factor. In other embodiments, the affinity of the binding of a growth factor is increased by the ALK activator. In specific embodiments, the growth factor is pleitotrophin. In other specific embodiments, the growth factor is midkine.

In certain embodiments, the ALK activator is capable of binding to the N-terminal domain of ALK, for example, to a region within amino acid residues 21-263 of SEQ ID NO:1. In a particular embodiment, the ALK activator is capable of binding within the positively charged region of the N-terminal domain of ALK. In one embodiment, the ALK activator is capable of binding to the heparin binding motif of ALK, e.g., human ALK, for example, to a region within amino acid residues 44-69 of SEQ ID NO: 1, for example amino acid residues 48-65 of SEQ ID NO:1.

In certain embodiments, the ALK activator is capable of binding to the N-terminal domain of ALK with a K_{D} of less than or equal to 0.25 μM. In specific embodiments, the ALK activator is capable of binding to the N-terminal domain of ALK with a K_{D} of less than or equal to 0.20, 0.15, 0.10, or 0.5 μM. In more specific embodiments, the ALK activator is capable of binding to the N-terminal domain of ALK with a K_{D} of less than or equal to 40, 30, 20, 10, 1.0, 0.5, 0.1 or 0.05 nM. In certain embodiments, the ALK activator is capable of binding to the N-terminal domain of ALK with a K_{D} of 1 pM to 100 nM, 10 pM to 50 nM, 10 pM to 10 nM, 10 pM to 1 nM, or 10 pM to 100 pM. The K_{D} can measured, for example, by surface plasmon resonance, isothermal titration calorimetry, or another method known to one of skill in the art.

In certain embodiments, the ALK activator is capable of binding to the heparin binding motif of ALK with a K_{D} of less than or equal to 0.25 μM. In specific embodiments, the ALK activator is capable of binding to the heparin binding motif of ALK with a K_{D} of less than or equal to 0.20, 0.15, 0.10, or 0.5 μM. In more specific embodiments, the ALK activator is capable of binding to the heparin binding motif of ALK with a K_{D} of less than or equal to 100, 40, 30, 20, 10, 1.0, 0.5, 0.1 or 0.05 nM. In certain embodiments, the ALK activator is capable of binding to the heparin binding motif of ALK with a K_{D} of 1 pM to 100 nM, 10 pM to 50 nM, 10 pM to 10 nM, 10 pM to 1 nM, or 10 pM to 100 pM.

In certain embodiments, the ALK activator comprises multiple (e.g., two, three, four, or more) ALK binding sites. In specific embodiments, the ALK activator comprises multiple ALK binding sites, wherein at least one ALK binding site binds a positively charged region of ALK, e.g., binds the N-terminal domain of ALK, for example within or overlapping with the heparin-binding region of ALK, and at least one ALK binding site binds a separate region of ALK, e.g., a separate region of the ALK extracellular domain. In one embodiment, the separate region of the ALK extracellular domain is within the MAM1 domain. In another embodiment, the separate region of the ALK extracellular domain is within the LDLa domain. In still another embodiment, the separate region of the ALK extracellular domain is within the MAM2 domain. In particular embodiments, the binding of the ALK activator to ALK can be inhibited by sucrose-octasulfate (SOS) or by a monomeric sulfated glycosaminoglycan.

In certain embodiments, the ALK activator is negatively charged at neutral pH. It is noted that unless otherwise specified, “negatively charged,” “positively charged,” “net negative charge,” “net positive charge,” and the like refer to charge at neutral pH. In a specific embodiment, an ALK activator comprises a negatively charged carbohydrate, e.g., a sulfated carbohydrate. In a particular embodiment, the ALK activator comprises dextran sulfate. In another embodiment, an ALK activator comprises an oligosaccharide or polysaccharide, e.g., a negatively charged oligosaccharide or polysaccharide, for example, a sulfated oligosaccharide or polysaccharide. In yet another embodiment, an ALK activator comprises a glycosaminoglycan, e.g., a negatively charged glycosaminoglycan, for example, a sulfated glycosaminoglycan. In another embodiment, such an ALK activator comprises a proteoglycan, e.g., a negatively charged proteoglycan, for example, a sulfated proteoglycan.

In certain embodiments, the ALK activator comprises an oligosaccharide (generally comprising ten or fewer monosaccharide units) or polysaccharide, e.g., a negatively charged oligosaccharide or polysaccharide. In specific embodiments, the oligosaccharide or polysaccharide has a chain length of at least 10, 15, 20, 25, 35, or at least 45 monosaccharides. The chain length of an ALK activator can be determined, for example, by isothermal titration calorimetry, or by any method known to one of skill in the art.

In certain embodiments, the ALK activator comprises an unbranched oligosaccharide or polysaccharide. In other embodiments, the ALK activator comprises a branched oligosaccharide or polysaccharide. In specific embodiments, the oligosaccharide or polysaccharide has a degree of branching of 2%, 3%, 4%, 5%, 6%, 7%, 8%, 9%, 10%, 12%, 14%, 16%, 18%, or 20%, wherein the degree of branching indicates the percentage of branch linkages per the total number of monosaccharide units. In a more specific embodiments, the oligosaccharide or polysaccharide has a degree of branching between 3 and 16%, or between 3 and 8%. In certain embodiments, the ALK activator comprises an oligosaccharide or polysaccharide that comprises O-glycosidic, N-glycosidic or S-glycosidic bonds, or a mixture thereof. In certain embodiments, the ALK activator comprises an oligosaccharide or polysaccharide that comprises α-linkages, β-linkages, or a mixture thereof.

In certain embodiments, the ALK activator comprises a negatively charged oligosaccharide or polysaccharide that has a charge density of between 0.1 and 6 equivalents per mole of monosaccharide at pH 7.0. As used herein, the charge of one equivalent is equal to the charge of one mole of electrons. For example, the charge of one mole of SO₄²⁻ is two equivalents. In certain embodiments, the negatively charged oligosaccharide or polysaccharide has a charge density of about 0.1, 0.5, 1, 2, 3, 4, 5, or 6 equivalents per mole. In certain embodiments, the oligosaccharide or polysaccharide is of uniform negative charge. In specific embodiments, the ALK activator comprises an oligosaccharide or polysaccharide that comprises a stretch of at least 10 monosaccharide units wherein the variation in charge does not exceed the charge of one electron. For example, if the average charge of that sequence of monosaccharide units is −2, then the variation in charge is from −1 to −3. If the average charge of that sequence of monosaccharide units is −3, then the variation in charge is from −2 to −4. If the average charge of that sequence of monosaccharide units is −5, then the variation in charge is from −4 to −6.

In certain embodiments, the ALK activator comprises an oligosaccharide or polysaccharide that comprises a sequence of at least 10 monosaccharide units wherein each monosaccharide is substituted with at least one negatively charged group, e.g., at least one sulfate group. In certain embodiments, the oligosaccharide or polysaccharide comprises a sequence of at least 10 monosaccharide units wherein each monosaccharide is substituted with at least two negatively charged groups, e.g., at least two sulfate groups. In other embodiments, the oligosaccharide or polysaccharide comprises a sequence of at least 10 monosaccharide units wherein each monosaccharide is substituted with at least three negatively charged groups, e.g., at with at least three sulfate groups. In yet other embodiments, the oligosaccharide or polysaccharide comprises a sequence of at least 10 monosaccharide units wherein each monosaccharide is substituted with at least four, five, six, or at least seven negatively charged groups, e.g., at least four, five, six, or at least seven sulfate groups.

In certain embodiments, the ALK activator comprises a glycosaminoglycan, e.g. a negatively charged glycosaminoglycan. In particular embodiments, the glycosaminoglycan comprises N-acetylglucosamine, N-acetylgalactosamine, or a mixture thereof. In other particular embodiments, the glycosaminoglycan comprises glucuronic acid, iduronic acid, galactose, or a mixture thereof. In still other embodiments, the glycosaminoglycan is a heparin/heparan sulfate (HSGAG), chondroitin/dermatan sulfate (CSGAGs), keratan sulfate, or hyaluronic acid glycosaminoglycan. Heparin and heparan sulfate, for example, can be distinguished, e.g., by the pattern of N-sulfation and N-acetylation, with heparan sulfate containing segregated sequences of N-sulfated and N-acetylated monosaccharides, and heparin having a greater number of solitary N-acetylated monosacharides and a greater number of N-sulfated monosaccharides in total (Gallagher and Walker, 1985, Biochem J. 230:665-674).

In certain embodiments, the ALK activator comprises a glycosaminoglycan with a chain length of at least 15 disaccharides. In certain embodiments, the ALK activator comprises a glycosaminoglycan with a chain length of at least 20 disaccharides. In certain embodiments, the ALK activator comprises a glycosaminoglycan with a chain length of at least 25 disaccharides.

In specific embodiments, the ALK activator comprises a glycosaminoglycan that has at most 1, 2, 3, 4, 5, 6, 7, 8, 9, 10, 11, 12, 13, 14, 15, 16, 17, 18, 19, 20, 21, 22, 23, 24, or 25 N-acetylated disaccharides per 100 disaccharide units. In other specific embodiments, the glycosaminoglycan is free or substantially free of N-acetylated disaccharides. In particular embodiments, the glycosaminoglycan has at least 50, 60, 70, 80, 90, or at least 100 N-sulfate groups per 100 disaccharide units. In specific embodiments, the glycosaminoglycan has at least 50, 60, 70, 80, 90, 100, 110, 120, 130, 140, or at least 150 O-sulfate groups per 100 disaccharide units. In specific embodiments, the glycosaminoglycan has at least 10, 20, 30, 40, 50, 60, 70, 80, or at least 90 trisulfated disaccharides per 100 disaccharide units. In more specific embodiments, substantially all of the disaccharides in the glycosaminoglycan are trisulfated.

In certain embodiments, the ALK activator comprises a sulfated glycosaminoglycan. In specific embodiments, the sulfated glycosaminoglycan is heparin. In certain embodiments, the ALK activator comprises an oversulfated glycosaminoglycan. “Oversulfation,” as used herein, refers to the synthetic addition of sulfates to a naturally occurring polysaccharide.

In certain embodiments, the ALK activator comprises a proteoglycan, e.g., a negatively charged proteoglycan, for example, a sulfated proteoglycan, wherein the proteoglycan comprises a protein or peptide attached to a carbohydrate, e.g., an oligosaccharide or polysaccharide. In more specific embodiments, the sulfated proteoglycan is a heparin proteoglycan. In other more specific embodiments, the sulfated proteoglycan is a chondroitin sulfate proteoglycan. In other more specific embodiments, the sulfated proteoglycan is a dermatan sulfate proteoglycan. In certain embodiments, the carbohydrate binds to the positively charged region of the N-terminal domain of ALK, for example, the heparin binding motif of the ALK receptor, wherein the peptide or protein binds to another region in the ALK extracellular domain.

In certain embodiments, the ALK activator associates with or binds to ALK. In another embodiment, the ALK activator associates with or binds to a polypeptide other than ALK. In yet another embodiment, the ALK activator associates with or binds to ALK and at least one other polypeptide, for example, at least one other polypeptide that associates with or binds to ALK. In a particular embodiment, the ALK activator associates or binds to a growth factor, e.g. midkine or pleiotrophin.

In certain embodiments, the ALK activator comprises an antibody. In other embodiments, the ALK activator comprises an antibody linked or conjugated, directly or indirectly, to an oligosaccharide or polysaccharide, e.g. a negatively charged oligosaccharide or polysaccharide. In specific embodiments, the oligosaccharide or polysaccharide binds to the N-terminal domain of ALK, e.g., the positively charged region of the N-terminal domain of ALK, for example, the heparin binding motif of ALK, while the antibody binds to another region in the ALK extracellular domain.

In certain embodiments, an ALK activator comprises a population of molecules that, on average, exhibit a structural characteristic of an ALK activator, as described herein. For example, in one embodiment, an ALK activator comprises a population of molecules that comprise carbohydrates, oligosaccharides, polysaccharides, glycosaminoglycans, or proteoglycans that, on average, exhibit a structural characteristic, e.g., a chain length, monosaccharide content, charge, etc. as described herein.

In certain embodiments, an ALK activator comprises a synthetically produced carbohydrate. In specific embodiments, the synthetically produced carbohydrate is an oligosaccharide or polysaccharide. In more specific embodiments, the synthetically produced oligosaccharide or polysaccharide is negatively charged. In other more specific embodiments, the synthetically produced oligosaccharide or polysaccharide is sulfated.

In another aspect, provided herein are pharmaceutical compositions of ALK activators. In particular aspects, compositions (e.g., pharmaceutical compositions) described herein can be used to induce or enhance ALK activity in order to manage or treat a disease or disorder, such as a neurodegenerative disease or disorder. In specific embodiments, provided herein is a pharmaceutical composition comprising an ALK activator described herein and a pharmaceutically acceptable carrier.

As used herein, the term “pharmaceutically acceptable” means being approved by a regulatory agency of the Federal or state government, or listed in the U.S. Pharmacopeia, European Pharmacopeia or other generally recognized Pharmacopeia for use in animals, or more particularly humans.

Therapeutic formulations containing one or more ALK activators provided herein can be prepared for storage by mixing the ALK activator having the desired degree of purity with optional physiologically acceptable carriers, excipients, or stabilizers in the form of lyophilized formulations or aqueous solutions (Remington's Pharmaceutical Sciences (1990) Mack Publishing Co., Easton, Pa.; Remington: The Science and Practice of Pharmacy, 21^{st} ed. (2006) Lippincott Williams & Wilkins, Baltimore, Md.). Acceptable carriers, excipients or stabilizers are nontoxic to recipients at the dosages and concentrations employed, and include buffers such as phosphate, citrate, and other organic acids; and/or non-ionic surfactants such as TWEEN™ PLURONICS™, or polyethylene glycol (PEG).

Formulations, such as those described herein, can also contain more than one active compound as necessary for the particular indication being treated. In certain embodiments, formulations comprise an ALK activator provided herein and one or more active compounds with complementary activities that do not adversely affect each other. Such molecules are suitably present in combination in amounts that are effective for the purpose intended. For example, an ALK activator described herein can be combined with one or more other therapeutic agents (including another ALK activator described herein). Such combination therapy can be administered to the patient serially or simultaneously in sequence.

The formulations to be used for in vivo administration can be sterile. This is readily accomplished through, e.g., sterile filtration membranes.

In specific aspects, the pharmaceutical compositions provided herein contain therapeutically effective amounts of one or more of the ALK activators provided herein, and optionally one or more additional prophylactic or therapeutic agents, in a pharmaceutically acceptable carrier. Such pharmaceutical compositions are useful in the prevention, treatment, management or amelioration of a neurodegenerative disease or disorder or one or more symptoms thereof.

Pharmaceutical carriers suitable for administration of the antibodies provided herein include any such carriers known to those skilled in the art to be suitable for the particular mode of administration.

Compositions provided herein can contain one or more ALK activators provided herein. In one embodiment, the ALK activators are formulated into suitable pharmaceutical preparations, such as solutions, suspensions, powders, sustained release formulations or elixirs in sterile solutions or suspensions for parenteral administration, or as transdermal patch preparation and dry powder inhalers.

The concentration of the ALK activator or activators in the compositions provided herein can be effective for delivery of an amount, upon administration, that treats, prevents, or ameliorates a neurodegenerative disease or disorder described herein or a symptom thereof. In one embodiment, compositions provided herein are formulated for single dosage administration. To formulate a composition, the weight fraction of compound is dissolved, suspended, dispersed, or otherwise mixed in a selected carrier at an effective concentration such that the treated condition is relieved, prevented, or one or more symptoms are ameliorated. In certain aspects, an ALK activator provided herein is included in the pharmaceutically acceptable carrier in an effective amount sufficient to exert a therapeutically useful effect in the absence of, or with minimal or negligible, undesirable side effects on the patient treated.

Pharmaceutical compositions described herein are provided for administration to humans or animals (e.g., mammals) in unit dosage forms, such as sterile parenteral (e.g., intravenous) solutions or suspensions containing suitable quantities of the compounds. Pharmaceutical compositions are also provided for administration to humans and animals in unit dosage form, such as tablets, capsules, pills, powders, granules, and oral or nasal solutions or suspensions, and oil-water emulsions containing suitable quantities of an ALK activator. The ALK activator is, in one embodiment, formulated and administered in unit-dosage forms or multiple dosage forms.

In certain embodiments, one or more ALK activators described herein are in a liquid pharmaceutical formulation. Liquid pharmaceutically administrable compositions can, for example, be prepared by dissolving, dispersing, or otherwise mixing an antibody and optional pharmaceutical adjuvants in a carrier, such as, for example, water, saline, aqueous dextrose, glycerol, glycols, and the like, to thereby form a solution or suspension. In certain embodiments, a pharmaceutical composition provided herein to be administered can also contain minor amounts of nontoxic auxiliary substances such as wetting agents, emulsifying agents, solubilizing agents, and pH buffering agents and the like.

Parenteral administration, in one embodiment, is characterized by injection, either subcutaneously, intramuscularly or intravenously is also contemplated herein. Injectables can be prepared in conventional forms, either as liquid solutions or suspensions, solid forms suitable for solution or suspension in liquid prior to injection, or as emulsions. The injectables, solutions and emulsions also contain one or more excipients. Suitable excipients are, for example, water, saline, dextrose, glycerol, or ethanol. Other routes of administration may include, enteric administration, intracerebral administration or intraventricular administration, nasal administration, intraarterial administration, intracardiac administration, intraosseous infusion, intrathecal administration, and intraperitoneal administration.

Preparations for parenteral administration include sterile solutions ready for injection, sterile dry soluble products, such as lyophilized powders, ready to be combined with a solvent just prior to use, including hypodermic tablets, sterile suspensions ready for injection, sterile dry insoluble products ready to be combined with a vehicle just prior to use and sterile emulsions. The solutions can be either aqueous or nonaqueous.

If administered intravenously, suitable carriers include physiological saline or phosphate buffered saline (PBS), and solutions containing thickening and solubilizing agents, such as glucose, polyethylene glycol, and polypropylene glycol and mixtures thereof.

Pharmaceutically acceptable carriers used in parenteral preparations include aqueous vehicles, nonaqueous vehicles, antimicrobial agents, isotonic agents, buffers, antioxidants, local anesthetics, suspending and dispersing agents, emulsifying agents, sequestering or chelating agents and other pharmaceutically acceptable substances.

Pharmaceutical carriers also include ethyl alcohol, polyethylene glycol and propylene glycol for water miscible vehicles; and sodium hydroxide, hydrochloric acid, citric acid or lactic acid for pH adjustment.

In other embodiments, the pharmaceutical formulations are lyophilized powders, which can be reconstituted for administration as solutions, emulsions and other mixtures. They can also be reconstituted and formulated as solids or gels. The lyophilized powder is prepared by dissolving an ALK activator provided herein in a suitable solvent. Suitable solvents can contain an excipient which improves the stability or other pharmacological component of the powder of reconstituted solution, prepared from the powder. Excipients that can be used include, but are not limited to, dextrose, sorbitol, fructose, corn syrup, xylitol, glycerin, glucose, sucrose or other suitable agent. A suitable solvent can also contain a buffer, such as citrate, sodium or potassium phosphate or other such buffer known to those of skill in the art at, in one embodiment, about neutral pH. Subsequent sterile filtration of the solution followed by lyophilization under standard conditions known to those of skill in the art provides an example of a formulation.

In certain aspects, ALK activators provided herein can be formulated for local administration or topical application, such as for topical application to the skin and mucous membranes, such as in the eye, in the form of gels, creams, and lotions and for application to the eye or for intracisternal or intraspinal application. Nasal solutions of the active compound alone or in combination with other pharmaceutically acceptable excipients can also be administered.

ALK activators provided herein can be formulated to be targeted to a particular tissue, receptor, or other area of the body of the subject to be treated. In some embodiments, ALK activators described herein are targeted (or otherwise administered) to the central or peripheral nervous system. In specific embodiments, an ALK activator described herein is capable of crossing the blood-brain barrier.

In certain aspects, ALK activators provided herein are formulated for administration to the central or peripheral nervous system. In certain embodiments, ALK activators provided herein are formulated to be transported across the blood-brain barrier via a specific carrier system or receptor-mediated endocytosis system. In other embodiments, ALK activators provided herein are formulated for administration in an intraparenchymal injection. In other embodiments, ALK activators provided herein are formulated for administration via an implantable system. In specific embodiments, the implantable system is an osmotic pump or enhanced convention device. In other embodiments, ALK activators provided herein are formulated for administration via intraventricular or intrathecal administration. In other embodiments, ALK activators provided herein are formulated for administration via intranasal delivery. In other embodiments, ALK activators provided herein are formulated for administration by transient disruption of the blood-brain barrier. In specific embodiments, transient disruption of the blood brain barrier is achieved by osmotic shock using mannitol, arabinose, or other hypertonic solution. In other embodiments, ALK activators provided herein are formulated for administration by microbubbles using magnetic resonance imaging-guided ultrasound.

In certain embodiments, ALK activators provided herein are formulated for administration using chemical derivatives to modify their structure to facilitate crossing of the blood-brain barrier. In other embodiments, ALK activators provided herein are formulated for administration using nanoparticle transporters.

In certain aspects, the ALK activators provided herein are formulated specifically to improve absorption. In some embodiments, the ALK activators provided herein are formulated to increase their lipophilicity. In some embodiments, the ALK activators provided herein are formulated to decrease their net charge. In specific embodiments, the ALK activators provided herein are formulated to increase their lipophilicity and decrease their net charge. In more specific embodiments, the ALK activators provided herein are formulated for administration within a positively charged liposome. In further specific embodiments, the positively charged liposome is a component of a pharmaceutical composition suitable for intranasal administration. In certain embodiments, the ALK activators provided herein are formulated for administration with a positively charged, non-active ingredient. In specific embodiments, the positively charged, non-active ingredient comprises a polyethylenimine. In other specific embodiments, the positively-charged, non-active ingredient makes up a part of a pharmaceutical composition suitable for intranasal administration.

### 5.2 Methods of Treating and/or Preventing Neurodegenerative, Neuromuscular and Cognitive Disorders

In one aspect, provided herein are methods of treating and/or preventing a neurodegenerative or neurological disease or disorder in a subject, e.g. a human, comprising administering to a subject in need thereof a therapeutically effective amount of an ALK activator, as described herein. In another aspect, provided herein are methods of treating and/or preventing a neuromuscular disease or disorder in a subject, e.g. a human, comprising administering to a subject in need thereof a therapeutically effective amount of an ALK activator, as described herein. In yet another aspect, provided herein are methods of treating and/or preventing a cognitive disease or disorder in a subject, e.g. a human, comprising administering to a subject in need thereof a therapeutically effective amount of an ALK activator, as described herein. In still another aspect, provided herein are methods of enhancing cognitive abilities in a subject, e.g. a human, comprising administering to the subject a therapeutically effective amount of an ALK activator, as described herein.

In one embodiment, the disease or disorder is Alzheimer's Disease. In another embodiment, the disease or disorder is post-traumatic stress disorder. In another embodiment, the disease or disorder is attention deficit disorder. In another embodiment, the disease or disorder is caused or exacerbated by substance abuse. In another embodiment, the disease or disorder is anxiety. In another embodiment, the disease or disorder is amyotrophic lateral sclerosis (ALS). In other embodiments, the disease or disorder is Huntington's disease, Parkinson's disease, dementia, an extrapyramidal disorder, a motor neuron disease, a systemic atrophy of the central nervous system, Tay-Sachs disease, ataxia telangiectasia, autosomal dominant cerebellar ataxia, Batten disease, corticobasal degeneration, Creutzfeldt-Jakob disease, Lyme disease, Machado-Joseph disease, cerebellar hypoplasia, multiple system atrophy, neuroanthocytosis, Niemann-Pick disease, pontocerebellar hypoplasia, Shy-Drager syndrome, spinocerebellar ataxia, subacute combined degeneration of the spinal cord, subacute sclerosing panencephalitis, Tabes dorsalis, toxic encephalopathy, toxic leukoencephalopathy, or Wobbly hedgehog syndrome. In another embodiment, the disease or disorder is multiple sclerosis, myasthenia gravis, spinal muscular atrophy, or muscular dystrophy, e.g., Duchenne muscular dystrophy or Becker muscular dystrophy. In another embodiment, the disease or disorder is microcephaly or hydrocephalus.

### 5.3 Inhibitors of ALK

In one aspect, presented herein are ALK regulators that are inhibitors of ALK tyrosine kinase activity, wherein such ALK inhibitors inhibit binding of heparin to the ALK receptor. The ability of an ALK inhibitor to inhibit binding of heparin to the ALK receptor can be tested using surface plasmon resonance. Specifically, heparin can be immobilized on a surface and the ability of the ALK extracellular domain to bind to the immobilized heparin is tested in the absence and in the presence of an ALK inhibitor. An illustrative assay is disclosed in Section 6.7.

In certain embodiments, the ALK inhibitor is capable of decreasing ALK tyrosine phosphorylation below background or of decreasing the level of ALK tyrosine phosphorylation induced by an ALK activator. For example, in one embodiment, the ALK inhibitor decreases ALK tyrosine phosphorylation below background in neuroblastoma cells cultured under normal cell culture conditions such as culturing in DMEM medium supplemented with 10% FBS and 1% penicillin/streptomycin. In certain embodiments, the neuroblastoma cells are Nagai, NB-39-nu, or NB-1 neuroblastoma cells.

In other embodiments, the ALK inhibitor decreases ALK tyrosine phosphorylation below background in cells, e.g., neuroblastoma cells, that express a constitutively active form of ALK. In specific embodiments, the constitutively active form of ALK contains a mutation in the kinase domain. In other embodiments, the ALK inhibitor decreases ALK tyrosine phosphorylation below background in cells, e.g., neuroblastoma cells, that overexpress ALK. In certain embodiments, 0.01 to 10 μM of the ALK inhibitor decreases ALK tyrosine phosphorylation below background in unstimulated cells, e.g., unstimulated neuroblastoma cells. In yet another embodiment, the ALK inhibitor decreases ALK tyrosine phosphorylation of stimulated cells, e.g., neuroblastoma cells that have been stimulated with an ALK activator (as disclosed in Section 5.1). In certain specific embodiments, the ALK inhibitor decreases ALK tyrosine phosphorylation of cells expressing an ALK receptor wherein the ALK receptor has been stimulated with an agonistic anti-ALK monoclonal antibody (see Section 6.2). Such ALK receptor expressing cells can be, for example, CHO cells that express the ALK receptor recombinantly. In certain embodiments, 0.01 to 10 μM of the ALK inhibitor decreases ALK tyrosine phosphorylation in unstimulated cells, e.g., unstimulated neuroblastoma cells. In another embodiment, 0.01 to 1.0 μM of the ALK inhibitor decreases ALK tyrosine phosphorylation in cells, e.g., neuroblastoma cells, that have been treated with 10 μg/mL of heparin.

In other embodiments, the ALK inhibitor decreases ALK tyrosine phosphorylation below background in cells that express a constitutively active form of ALK, cultured under normal cell culture conditions, e.g., as provided above. In other embodiments, the ALK inhibitor decreases ALK tyrosine phosphorylation below background in cells that overexpress ALK, cultured under normal cell culture conditions, e.g, as provided above. In specific embodiments, the cells that overexpress ALK are thyroid carcinoma, non-small cell lung cancer, breast cancer, melanoma, glioblastoma, astrocytoma, retinoblastoma, ewing sarcoma, or rhabdomyosarcoma cells. In more specific embodiments, the cells that overexpress ALK are glioblastoma cells.

In certain embodiments, the ALK inhibitor is present in a composition of ALK inhibitor molecules with varying molecular weights. In certain embodiments, the average molecular weight of the ALK inhibitor is 5 kDa, 10 kDa, 15 kDa, 20 kDa, or 25 kDa. In other embodiments, the median molecular weight of the ALK inhibitor is 5 kDa, 10 kDa, 15 kDa, 20 kDa, or 25 kDa. In certain embodiments, at least 50%, 60%, 70%, 80%, or at least 90% of the ALK inhibitor molecules in such a composition are within a margin of at most 10%, at most 15%, at most 20%, or at most 25% below and above the average molecular weight. In certain embodiments, at least 50%, 60%, 70%, 80%, or at least 90% of the ALK inhibitor molecules in such a composition are within a margin of at most 10%, at most 15%, at most 20%, or at most 25% below and above the median molecular weight. For example, if the average molecular weight is 15 kDa and the margin is 10%, then at least 50%, 60%, 70%, 80%, or at least 90% of the ALK inhibitor molecules in such a composition have a molecular weight between 13.5 kDa and 16.5 kDa.

In certain embodiments, 0.01 to 10 μM of the ALK inhibitor decreases ALK tyrosine phosphorylation in unstimulated neuroblastoma cells. In a specific embodiments, 1.0 μM of the ALK inhibitor decreases ALK tyrosine phosphorylation in unstimulated neuroblastoma cells. In other specific certain embodiments, 0.01, 0.05, 0.1, 0.5, 5, or 10 μM of the ALK inhibitor decreases ALK tyrosine phosphorylation in unstimulated neuroblastoma cells.

In certain embodiments, 0.01 to 10 μM of the ALK inhibitor decreases ALK tyrosine phosphorylation by 5%, 10%, 15%, 25%, 50%, or 100% in stimulated neuroblastoma cells. In a specific embodiments, 1.0 μM of the ALK inhibitor decreases ALK tyrosine phosphorylation by 5%, 10%, 15%, 25%, 50%, or 100% in stimulated neuroblastoma cells. In other specific certain embodiments, 0.01, 0.05, 0.1, 0.5, 5, or 10 μM of the ALK inhibitor decreases ALK tyrosine phosphorylation by 5%, 10%, 15%, 25%, 50%, or 100% in stimulated neuroblastoma cells.

In certain embodiments, 0.01 to 10 μM of the ALK inhibitor decreases ALK tyrosine phosphorylation 2, 3, 4, 5, 6, 7, 8, 9, or 10-fold in stimulated neuroblastoma cells. In a specific embodiment, 1.0 μM of the ALK inhibitor decreases ALK tyrosine phosphorylation 2, 3, 4, 5, 6, 7, 8, 9, or 10-fold in stimulated neuroblastoma cells. In other specific embodiments, 0.01, 0.05, 0.1, 0.5, 5, or 10 μM of the ALK inhibitor decreases ALK tyrosine phosphorylation 2, 3, 4, 5, 6, 7, 8, 9, or 10-fold in stimulated neuroblastoma cells.

In certain embodiments, the ALK inhibitor is capable of decreasing ALK tyrosine phosphorylation below background in any unstimulated ALK-expressing cell, e.g., an ALK-expressing cell or cell line, or a cell or cell line which has been transfected with an ALK vector to stably or transiently express ALK, known to one of skill in the art. In particular embodiments, 0.01 to 10 μM of the ALK inhibitor is capable of decreasing ALK tyrosine phosphorylation below background in an unstimulated ALK-expressing cell. In a specific embodiment, 1.0 μM of the ALK inhibitor is capable of decreasing ALK tyrosine phosphorylation below background in an unstimulated ALK-expressing cell. In other specific embodiments, 0.01, 0.05, 0.1, 0.5, 5, or 10 μM of the ALK inhibitor is capable of decreasing ALK tyrosine phosphorylation below background in an unstimulated ALK-expressing cell.

In certain embodiments, the ALK inhibitor is capable of decreasing ALK tyrosine phosphorylation in any stimulated ALK-expressing cell, e.g., an ALK-expressing cell or cell line, or a cell or cell line which has been transfected with an ALK vector to stably or transiently express ALK, known to one of skill in the art. In particular embodiments, 0.01 to 10 μM of the ALK inhibitor is capable of decreasing ALK tyrosine phosphorylation in a stimulated ALK-expressing cell. In a specific embodiment, 1.0 μM of the ALK inhibitor is capable of decreasing ALK tyrosine phosphorylation in a stimulated ALK-expressing cell. In other specific embodiments, 0.01, 0.05, 0.1, 0.5, 5, or 10 μM of the ALK inhibitor is capable of decreasing ALK tyrosine phosphorylation in a stimulated ALK-expressing cell.

In certain embodiments, 0.01 to 10 μM of the ALK inhibitor decreases ALK tyrosine phosphorylation by 5%, 10%, 15%, 25%, 50%, or 100% in a stimulated ALK-expressing cell. In a specific embodiment, 1.0 μM of the ALK inhibitor decreases ALK tyrosine phosphorylation by 5%, 10%, 15%, 25%, 50%, or 100% in a stimulated ALK-expressing cell. In other specific embodiments, 0.01, 0.05, 0.1, 0.5, 5, or 10 μM of the ALK inhibitor decreases ALK tyrosine phosphorylation by 5%, 10%, 15%, 25%, 50%, or 100% in a stimulated ALK-expressing cell. In certain embodiments, 0.01 to 10 μM of the ALK inhibitor decreases ALK tyrosine phosphorylation by 2, 3, 4, 5, 6, 7, 8, 9, or 10-fold in a stimulated ALK-expressing cell. In a specific embodiment, 1.0 μM of the ALK inhibitor decreases ALK tyrosine phosphorylation by 2, 3, 4, 5, 6, 7, 8, 9, or 10-fold in a stimulated ALK-expressing cell. In other specific embodiments, 0.01, 0.05, 0.1, 0.5, 5, or 10 μM of the ALK inhibitor decreases ALK tyrosine phosphorylation by 2, 3, 4, 5, 6, 7, 8, 9, or 10-fold in a stimulated ALK-expressing cell.

Tyrosine phosphorylation of ALK can be measured, for example, by an immunoblotting assay using an antibody that binds to phosphorylated ALK but not ALK that has not been phosphorylated (see, e.g., Moog-Lutz et al., 2005, J. Biol. Chem. 280(28):26039-26048). Phosphorylation of downstream targets of ALK can be measured, for example, by an immunoblotting assay using an antibody that binds to the phosphorylated target but not the target that has not been phosphorylated (see, e.g., Moog-Lutz et al., 2005). Any statistical method known to one of skill in the art can be used to confirm that the ALK tyrosine phosphorylation is significant and reproducible.

Various assays can be used to confirm the activity of an ALK inhibitor. In certain embodiments, 1.0 μM of the ALK inhibitor is capable of decreasing ALK tyrosine phosphorylation below background in any ALK-expressing cell line known to one of skill in the art or in decreasing ALK tyrosine phosphorylation in an ALK-expressing cell line in the presence of an ALK activator. In other embodiments, 0.01, 0.05, 0.1, 0.5, 5, or 10 μM of the ALK inhibitor is capable of decreasing ALK tyrosine phosphorylation below background in any ALK-expressing cell line known to one of skill in the art or in decreasing ALK tyrosine phosphorylation in an ALK-expressing cell line in the presence of an ALK activator. In other embodiments, the ALK inhibitor is capable of decreasing ALK tyrosine phosphorylation below background in a cell line which has been transfected with an ALK vector to stably or transiently express ALK. In certain embodiments, 1.0 μM of the ALK inhibitor decreases phosphorylation by 5%, 10%, 15%, 25%, 50%, or 100% in an unstimulated cell line stably or transiently expressing ALK or the same cell line in the presence of an ALK activator. In other embodiments, 1.0 μM of the ALK inhibitor decreases phosphorylation 2, 3, 4, 5, 6, 7, 8, 9, or 10-fold in an unstimulated cell line stably or transiently expressing ALK, or in the same cell line in the presence of an ALK activator.

In certain embodiments, the ALK inhibitor is capable of decreasing proliferation of cells stably or transiently expressing ALK in the presence of an ALK activator. In other embodiments, the ALK inhibitor is capable of decreasing cell proliferation in a cell line stably or transiently expressing constitutively active ALK. In certain embodiments, 0.01, 0.05, 0.1, 0.5, 1, 5, or 10 μM of the ALK inhibitor is capable of decreasing cell proliferation in a cell line stably or transiently expressing ALK in the presence of an ALK activator by at least 10%, 20%, 30%, 40%, 50%, 60%, 70%, 80%, 90%, 95%, at least 98%, or by 100%. In certain embodiments, 0.01, 0.05, 0.1, 0.5, 1, 5, or 10 μM of the ALK inhibitor is capable of decreasing cell proliferation in a cell line stably or transiently expressing constitutively active ALK by at least 10%, 20%, 30%, 40%, 50%, 60%, 70%, 80%, 90%, 95%, at least 98%, or by 100%.

In certain embodiments, the ALK inhibitor is capable of decreasing neurite outgrowth in PC 12 cells stably or transiently expressing ALK in the presence of an ALK activator. In other embodiments, the ALK inhibitor is capable of decreasing neurite outgrowth in a neuronal cell line stably or transiently expressing ALK in the presence of an ALK activator. In certain embodiments, 1.0 μM of the ALK inhibitor is capable of decreasing neurite outgrowth in a neuronal cell line stably or transiently expressing ALK in the presence of an ALK activator. In certain embodiments, 0.01, 0.05, 0.1, 0.5, 5, or 10 μM of the ALK inhibitor is capable of decreasing neurite outgrowth in a neuronal cell line stably or transiently expressing ALK in the presence of an ALK activator. In specific embodiments, 1.0 μM of the ALK inhibitor is capable of decreasing the percentage of cells extending neurites by 5%, 10%, 15%, 25%, 50%, or 100% in a neuronal cell line stably or transiently expressing ALK in the presence of an ALK activator. In other specific embodiments, 1.0 μM of the ALK inhibitor is capable of decreasing the percentage of cells extending neurites 2, 3, 4, 5, 6, 7, 8, 9, or 10-fold in a neuronal cell line stably or transiently expressing ALK in the presence of an ALK activator.

In certain embodiments, the ALK inhibitor is capable of decreasing ERK1/2 phosphorylation in a cell line stably or transiently expressing ALK in the presence of an ALK activator. In other embodiments, the ALK inhibitor is capable of decreasing AKT phosphorylation in a cell line stably or transiently expressing ALK in the presence of an ALK activator. In other embodiments, the ALK inhibitor is capable of decreasing STAT1 phosphorylation in a cell line stably or transiently expressing ALK in the presence of an ALK activator. In other embodiments, the ALK inhibitor is capable of decreasing STAT3 phosphorylation in a cell line stably or transiently expressing ALK in the presence of an ALK activator. In other embodiments, the ALK inhibitor is capable of decreasing STAT5 phosphorylation in a cell line stably or transiently expressing ALK in the presence of an ALK activator. In specific embodiments, 1.0 μM of the ALK inhibitor is capable of decreasing phosphorylation in one or more of ERK1/2, AKT, STAT1, STAT3, and STAT5 in the presence of an ALK activator. In specific embodiments, 0.01, 0.05, 0.1, 0.5, 5, or 10 μM of the ALK inhibitor is capable of decreasing phosphorylation in one or more of ERK1/2, AKT, STAT1, STAT3, and STAT5 in the presence of an ALK activator. In specific embodiments, 1.0 μM of the ALK inhibitor is capable of decreasing phosphorylation in one or more of ERK1/2, AKT, STAT1, STAT3, and STAT5 by 5%, 10%, 15%, 25%, 50%, or 100% in a cell line stably or transiently expressing ALK in the presence of an ALK activator. In other specific embodiments, 1.0 μM of the ALK inhibitor is capable of decreasing phosphorylation 2, 3, 4, 5, 6, 7, 8, 9, or 10-fold in one or more of ERK1/2, AKT, STAT1, STAT3, and STAT5 in a cell line stably or transiently expressing ALK in the presence of an ALK activator.

In certain embodiments, the ALK inhibitor is capable of decreasing the oligomerization of ALK or the ALK extracellular domain. In specific embodiments, the ALK inhibitor is capable of decreasing the oligomerization of ALK or the ALK extracellular domain as measured by SEC-MALLS. In other specific embodiments, the ALK inhibitor is capable of decreasing the dimerization of ALK or the ALK extracellular domain as measured by SEC-MALLS. In other specific embodiments, the ALK inhibitor is capable of decreasing the tetramerization of the ALK extracellular domain as measured by SEC-MALLS. In other specific embodiments, the ALK inhibitor is capable of decreasing the pentamerization of the ALK extracellular domain as measured by SEC-MALLS. In specific embodiments, the ALK inhibitor is capable of decreasing the oligomerization of ALK or the ALK extracellular domain as measured by isothermal titration calorimetry. In more specific embodiments, the ALK inhibitor is capable of decreasing the dimerization of ALK or the ALK extracellular domain by 10, 20, 30, 40, 50, or 100% as measured by SEC-MALLS. In more specific embodiments, the ALK inhibitor is capable of decreasing the dimerization of ALK or the ALK extracellular domain by 10, 20, 30, 40, 50, or 100% as measured by isothermal titration calorimetry.

In certain embodiments, the ALK inhibitor inhibits binding of an oligosaccharide or polysaccharide described above (e.g., heparin) to ALK. In specific embodiments, the ALK inhibitor inhibits binding of an oligosaccharide or polysaccharide described above (e.g., heparin) to the N-terminal domain of ALK. In certain embodiments, the ALK inhibitor inhibits binding of an oligosaccharide or polysaccharide described above (e.g., heparin) to the heparin binding motif of ALK. In certain embodiments, the heparin binding motif comprises amino acid residues 48 to 65 of human ALK. In specific embodiments, the heparin binding motif comprises residues 48 to 65 of SEQ ID NO: 1. The binding of the ALK inhibitor to ALK can be measured, for example, by isothermal titration calorimetry, surface plasmon resonance, or other methods known to one of skill in the art.

In certain embodiments, the ALK inhibitor reduces binding of an oligosaccharide or polysaccharide described above (e.g., heparin) to the ALK receptor as measured by a surface plasmon resonance dissociation assay. In certain embodiments, the ALK inhibitor reduces binding of an oligosaccharide or polysaccharide described above (e.g., heparin) to ALK as measured by a surface plasmon resonance competition assay. In other embodiments, the ALK inhibitor reduces binding of an oligosaccharide or polysaccharide described above (e.g., heparin) to ALK as measured by a reduction in heparin-induced ALK tyrosine phosphorylation in a cell line stably or transiently expressing ALK. In other embodiments, the ALK inhibitor reduces binding of an oligosaccharide or polysaccharide described above (e.g., heparin) to ALK as measured by solid state inhibition of binding of a labeled oligosaccharide or polysaccharide described above (e.g., heparin) to immobilized ALK. In other embodiments, the ALK inhibitor reduces binding of an oligosaccharide or polysaccharide described above (e.g., heparin) to ALK as measured by solid state inhibition of binding of labeled ALK to an immobilized oligosaccharide or polysaccharide described above (e.g., heparin).

In certain embodiments, the ALK inhibitor inhibits binding of an oligosaccharide or polysaccharide described above (e.g., heparin) to ALK with an IC₅₀ of less than or equal to 0.5 μM. In specific embodiments, the ALK inhibitor inhibits binding of an oligosaccharide or polysaccharide described above (e.g., heparin) to ALK with an IC₅₀ of less than or equal to 0.4, 0.3, 0.2, or less than or equal to 0.1 μM. In other specific embodiments, the ALK inhibitor inhibits binding of an oligosaccharide or polysaccharide described above (e.g., heparin) to ALK with an IC₅₀ of less than or equal to 50, 40, 30, 20, 10 or less than or equal to 1.0 nM. In other specific embodiments, the ALK inhibitor inhibits binding of an oligosaccharide or polysaccharide described above (e.g., heparin) to ALK with an IC₅₀ of less than or equal to 500, 100, 50, 40, 30, 20, or less than or equal to 10 pM. In particular embodiments, the ALK inhibitor inhibits binding of an oligosaccharide or polysaccharide described above (e.g., heparin) to ALK with an IC₅₀ of less than or equal to 10 pM to 0.5 μM, 100 pM to 100 nM, or 100 pM to 1 nM. In certain embodiments, the IC₅₀ is measured by surface plasmon resonance. In other embodiments, the IC₅₀ is measured by a solid-phase assay.

In certain embodiments, the ALK inhibitor binds to ALK. In specific embodiments, the ALK inhibitor binds to the N-terminal domain of ALK. In certain embodiments, the ALK inhibitor binds to the heparin binding motif of ALK. In specific embodiments, the ALK inhibitor binds to a region within amino acid residues 44-69 of human ALK. In more specific embodiments, the ALK inhibitor binds to a region within amino acid residues 44-69 of SEQ ID NO:1, for example, amino acid residues 48 to 65 of SEQ ID NO:1.

In certain embodiments, the ALK inhibitor does not inhibit binding of pleiotrophin to ALK. In certain embodiments, the ALK inhibitor does not inhibit binding of midkine to ALK.

In specific embodiments, the ALK inhibitor is an antibody that binds specifically to ALK. In other specific embodiments, the ALK inhibitor is a soluble protein that comprises the heparin binding motif of ALK, for example, a region within amino acids 44-49, e.g., amino acids 48 to 65 of human ALK. In more specific embodiments, the ALK inhibitor is a soluble protein that comprises a heparin binding portion ALK, for example, a heparin binding portion of the N-terminal domain of ALK, e.g., the heparin binding motif of ALK, for example human ALK, e.g., amino acids 48 to 65 of SEQ ID NO:1, or a heparin binding portion thereof.

In certain embodiments, the ALK inhibitor binds to an ALK activator. In certain embodiments, the ALK inhibitor is a positively charged carbohydrate. In certain embodiments, the ALK inhibitor is a positively charged oligosaccharide or polysaccharide. In other embodiments, the ALK inhibitor is a positively charged proteoglycan. In other embodiments, the ALK inhibitor is a carbohydrate-binding protein with a net positive charge. In specific embodiments, the ALK inhibitor comprises a lectin. In other specific embodiments, the ALK inhibitor comprises a glycosaminoglycan-binding protein. In a specific embodiment, the ALK inhibitor comprises a sulfated-glycosaminoglycan-binding protein. In more specific embodiments, the ALK inhibitor comprises a C-type, P-type, I-type, L-type, or R-type lectin or galectin. In more specific embodiments, the lectin is Anadarin MS or another heparin-binding lectin. In certain embodiments, the lectin binds to heparin but not to heparan sulfate.

In certain embodiments, the ALK inhibitor comprises a soluble protein. In particular embodiments, the soluble protein comprises an ALK, e.g., human ALK, extracellular domain or portion thereof. In still other embodiments, the soluble protein comprises an ALK, e.g., human ALK, N-terminal domain or a portion thereof. In a specific embodiment, the soluble protein comprises a positively charged region. In still other embodiments, the ALK inhibitor comprises a heparin binding motif, e.g., an ALK, for example, human ALK, heparin binding motif or a fibroblast growth factor heparin binding motif. In yet another embodiment, the soluble protein comprises a fibroblast growth factor domain.

In certain embodiments, the ALK inhibitor comprises an antibody. In a specific embodiment, the antibody inhibits heparin binding to ALK, e.g., human ALK. In a certain embodiment, the antibody inhibits binding of heparin to the N-terminal domain of ALK, e.g., human ALK. In specific embodiments, the ALK inhibitor binds to a region within amino acid residues 44-69 of human ALK. In more specific embodiments, the ALK inhibitor binds to a region within amino acid residues 44-69 of SEQ ID NO: 1, for example, amino acid residues 48 to 65 of SEQ ID NO: 1. In certain embodiments, the antibody is conjugated to a molecule, e.g., a toxin.

In another aspect, provided herein are pharmaceutical compositions of ALK inhibitors. In particular aspects, compositions (e.g., pharmaceutical compositions) described herein can be used to induce or enhance ALK activity in order to manage or treat a disease or disorder, such as a hyperproliferative or neoplastic disease or disorder, such as a cancer. In specific embodiments, provided herein is a pharmaceutical composition comprising an ALK inhibitor described herein and a pharmaceutically acceptable carrier.

As used herein, the term “pharmaceutically acceptable” means being approved by a regulatory agency of the Federal or state government, or listed in the U.S. Pharmacopeia, European Pharmacopeia or other generally recognized Pharmacopeia for use in animals, or more particularly humans.

Therapeutic formulations containing one or more ALK inhibitors provided herein can be prepared for storage by mixing the ALK inhibitor having the desired degree of purity with optional physiologically acceptable carriers, excipients, or stabilizers in the form of lyophilized formulations or aqueous solutions (Remington's Pharmaceutical Sciences (1990) Mack Publishing Co., Easton, Pa.; Remington: The Science and Practice of Pharmacy, 21^{st} ed. (2006) Lippincott Williams & Wilkins, Baltimore, Md.). Acceptable carriers, excipients or stabilizers are nontoxic to recipients at the dosages and concentrations employed, and include buffers such as phosphate, citrate, and other organic acids; and/or non-ionic surfactants such as TWEEN™ PLURONICS™, or polyethylene glycol (PEG).

Formulations, such as those described herein, can also contain more than one active compound as necessary for the particular indication being treated. In certain embodiments, formulations comprise an ALK inhibitor provided herein and one or more active compounds with complementary activities that do not adversely affect each other. Such molecules are suitably present in combination in amounts that are effective for the purpose intended. For example, an ALK inhibitor described herein can be combined with one or more other therapeutic agents (including another ALK inhibitor described herein). Such combination therapy can be administered to the patient serially or simultaneously in sequence.

The formulations to be used for in vivo administration can be sterile. This is readily accomplished through, e.g., sterile filtration membranes.

In specific aspects, the pharmaceutical compositions provided herein contain therapeutically effective amounts of one or more of the ALK inhibitors provided herein, and optionally one or more additional prophylactic or therapeutic agents, in a pharmaceutically acceptable carrier. Such pharmaceutical compositions are useful in the prevention, treatment, management or amelioration of a hyperproliferative or neoplastic disease or disorder, such as a cancer, or one or more symptoms thereof.

Pharmaceutical carriers suitable for administration of the antibodies provided herein include any such carriers known to those skilled in the art to be suitable for the particular mode of administration.

Compositions provided herein can contain one or more ALK inhibitors provided herein. In one embodiment, the ALK inhibitors are formulated into suitable pharmaceutical preparations, such as solutions, suspensions, powders, sustained release formulations or elixirs in sterile solutions or suspensions for parenteral administration, or as transdermal patch preparation and dry powder inhalers.

The concentration of the ALK inhibitor or inhibitors in the compositions provided herein can be effective for delivery of an amount, upon administration, that treats, prevents, or ameliorates a hyperproliferative or neoplastic disease or disorder, such as a cancer, described herein or a symptom thereof. In one embodiment, compositions provided herein are formulated for single dosage administration. To formulate a composition, the weight fraction of compound is dissolved, suspended, dispersed, or otherwise mixed in a selected carrier at an effective concentration such that the treated condition is relieved, prevented, or one or more symptoms are ameliorated. In certain aspects, an ALK inhibitor provided herein is included in the pharmaceutically acceptable carrier in an effective amount sufficient to exert a therapeutically useful effect in the absence of, or with minimal or negligible, undesirable side effects on the patient treated.

Pharmaceutical compositions described herein are provided for administration to humans or animals (e.g., mammals) in unit dosage forms, such as sterile parenteral (e.g., intravenous) solutions or suspensions containing suitable quantities of the compounds. Pharmaceutical compositions are also provided for administration to humans and animals in unit dosage form, such as tablets, capsules, pills, powders, granules, and oral or nasal solutions or suspensions, and oil-water emulsions containing suitable quantities of an ALK inhibitor. The ALK inhibitor is, in one embodiment, formulated and administered in unit-dosage forms or multiple dosage forms.

In certain embodiments, one or more ALK inhibitors described herein are in a liquid pharmaceutical formulation. Liquid pharmaceutically administrable compositions can, for example, be prepared by dissolving, dispersing, or otherwise mixing an antibody and optional pharmaceutical adjuvants in a carrier, such as, for example, water, saline, aqueous dextrose, glycerol, glycols, and the like, to thereby form a solution or suspension. In certain embodiments, a pharmaceutical composition provided herein to be administered can also contain minor amounts of nontoxic auxiliary substances such as wetting agents, emulsifying agents, solubilizing agents, and pH buffering agents and the like.

Parenteral administration, in one embodiment, is characterized by injection, either subcutaneously, intramuscularly or intravenously is also contemplated herein. Injectables can be prepared in conventional forms, either as liquid solutions or suspensions, solid forms suitable for solution or suspension in liquid prior to injection, or as emulsions. The injectables, solutions and emulsions also contain one or more excipients. Suitable excipients are, for example, water, saline, dextrose, glycerol, or ethanol. Other routes of administration may include, enteric administration, intracerebral administration, intraventricular administration, nasal administration, intraarterial administration, intracardiac administration, intraosseous infusion, intrathecal administration, and intraperitoneal administration.

Preparations for parenteral administration include sterile solutions ready for injection, sterile dry soluble products, such as lyophilized powders, ready to be combined with a solvent just prior to use, including hypodermic tablets, sterile suspensions ready for injection, sterile dry insoluble products ready to be combined with a vehicle just prior to use and sterile emulsions. The solutions can be either aqueous or nonaqueous.

If administered intravenously, suitable carriers include physiological saline or phosphate buffered saline (PBS), and solutions containing thickening and solubilizing agents, such as glucose, polyethylene glycol, and polypropylene glycol and mixtures thereof.

Pharmaceutically acceptable carriers used in parenteral preparations include aqueous vehicles, nonaqueous vehicles, antimicrobial agents, isotonic agents, buffers, antioxidants, local anesthetics, suspending and dispersing agents, emulsifying agents, sequestering or chelating agents and other pharmaceutically acceptable substances.

Pharmaceutical carriers also include ethyl alcohol, polyethylene glycol and propylene glycol for water miscible vehicles; and sodium hydroxide, hydrochloric acid, citric acid or lactic acid for pH adjustment.

In other embodiments, the pharmaceutical formulations are lyophilized powders, which can be reconstituted for administration as solutions, emulsions and other mixtures. They can also be reconstituted and formulated as solids or gels. The lyophilized powder is prepared by dissolving an ALK inhibitor provided herein in a suitable solvent. Suitable solvents can contain an excipient which improves the stability or other pharmacological component of the powder of reconstituted solution, prepared from the powder. Excipients that can be used include, but are not limited to, dextrose, sorbitol, fructose, corn syrup, xylitol, glycerin, glucose, sucrose or other suitable agent. A suitable solvent can also contain a buffer, such as citrate, sodium or potassium phosphate or other such buffer known to those of skill in the art at, in one embodiment, about neutral pH. Subsequent sterile filtration of the solution followed by lyophilization under standard conditions known to those of skill in the art provides an example of a formulation.

In certain aspects, ALK inhibitors provided herein can be formulated for local administration or topical application, such as for topical application to the skin and mucous membranes, such as in the eye, in the form of gels, creams, and lotions and for application to the eye or for intracisternal or intraspinal application. Nasal solutions of the active compound alone or in combination with other pharmaceutically acceptable excipients can also be administered.

ALK inhibitors provided herein can be formulated to be targeted to a particular tissue, receptor, or other area of the body of the subject to be treated. In some embodiments, ALK inhibitors described herein are targeted (or otherwise administered) to visual organs, bone marrow, gastrointestinal tract, lungs, brain, or joints. In specific embodiments, an ALK inhibitor described herein is capable of crossing the blood-brain barrier.

### 5.4 Methods of Treating Hyperproliferative Disorders

In one aspect, provided herein are methods of treating or preventing a disease or disorder associated with cells that express ALK. In one embodiment, provided herein are methods of treating or preventing a disease or disorder associated abnormal, e.g., elevated or constitutive, ALK tyrosine kinase activity, comprising administering to a subject in need thereof a therapeutically effective amount of an ALK inhibitor, wherein the ALK inhibitor inhibits binding of heparin to ALK.

In one embodiment, disease or disorder is a hyperproliferative disease or disorder. In certain embodiments, the hyperproliferative disease or disorder is cancer. In a specific embodiment, the cancer is lymphoma. In a more specific embodiment, the lymphoma is an anaplastic large-cell lymphoma. In other specific embodiments, the cancer is a non-small cell lung cancer, inflammatory breast cancer, medulloblastoma, rhabdomyosarcoma, colorectal cancer, pancreatic cancer, myofibroblastic tumors, Ewing's sarcomas, head-and-neck cancer, neurofibromatosis, ovarian cancer, or glioblastoma. In a more specific embodiment, the cancer is a neuroblastoma, for example, pediatric neuroblastoma

### 5.5 Methods of Screening for ALK Ligands

In certain embodiments, provided herein is a method of screening for an ALK ligand, comprising i) contacting an ALK-expressing cell with a test agent and heparin, and ii) measuring the level of ALK tyrosine phosphorylation; wherein an increase in ALK tyrosine phosphorylation in the presence of the test agent and heparin in comparison with the level of ALK tyrosine phosphorylation in the absence of the test agent indicates that the test agent is an ALK ligand. In certain embodiments, the ALK-expressing cell is a neuroblastoma cell. In specific embodiments, a 5% increase in ALK tyrosine phosphorylation in the presence of the test agent and heparin in comparison with the level of ALK tyrosine phosphorylation in the absence of the test agent indicates that the test agent is an ALK ligand. In specific embodiments, a 10% increase in ALK tyrosine phosphorylation in the presence of the test agent and heparin in comparison with the level of ALK tyrosine phosphorylation in the absence of the test agent indicates that the test agent is an ALK ligand. In specific embodiments, a 25% increase in ALK tyrosine phosphorylation in the presence of the test agent and heparin in comparison with the level of ALK tyrosine phosphorylation in the absence of the test agent indicates that the test agent is an ALK ligand. In specific embodiments, a 50% increase in ALK tyrosine phosphorylation in the presence of the test agent and heparin in comparison with the level of ALK tyrosine phosphorylation in the absence of the test agent indicates that the test agent is an ALK ligand. In specific embodiments, a 5%, 10%, 15%, 25%, 50%, 75%, or 100% increase in ALK tyrosine phosphorylation in the presence of the test agent and heparin in comparison with the level of ALK tyrosine phosphorylation in the absence of the test agent indicates that the test agent is an ALK ligand.

In certain embodiments, provided herein is a method of screening for an ALK activator, comprising i) contacting an ALK-expressing cell with a test agent and heparin, and ii) measuring the level of ERK 1/2 phosphorylation, wherein an increase in ERK 1/2 phosphorylation in the presence of the test agent and heparin in comparison with the level of ERK 1/2 phosphorylation in the absence of the test agent indicates that the test agent is an ALK activator. In certain embodiments, the ALK-expressing cell is a neuroblastoma cell. In specific embodiments, a 5% increase in ERK 1/2 phosphorylation in the presence of the test agent and heparin in comparison with the level of ERK 1/2 phosphorylation in the absence of the test agent indicates that the test agent is an ALK ligand. In specific embodiments, a 10% increase in ERK 1/2 phosphorylation in the presence of the test agent and heparin in comparison with the level of ERK 1/2 phosphorylation in the absence of the test agent indicates that the test agent is an ALK ligand. In specific embodiments, a 25% increase in ERK 1/2 phosphorylation in the presence of the test agent and heparin in comparison with the level of ERK 1/2 phosphorylation in the absence of the test agent indicates that the test agent is an ALK ligand. In specific embodiments, a 50% increase in ERK 1/2 phosphorylation in the presence of the test agent and heparin in comparison with the level of ERK 1/2 phosphorylation in the absence of the test agent indicates that the test agent is an ALK ligand. In specific embodiments, a 5%, 10%, 15%, 25%, 50%, 75%, or 100% increase in ERK 1/2 phosphorylation in the presence of the test agent and heparin in comparison with the level of ERK 1/2 phosphorylation in the absence of the test agent indicates that the test agent is an ALK ligand.

In certain embodiments, provided herein is a method of screening for an ALK activator, comprising i) contacting an ALK-expressing cell with a test agent and heparin, and ii) measuring the level of STAT3 phosphorylation, wherein an increase in STAT3 phosphorylation in the presence of the test agent and heparin in comparison with the level of STAT3 phosphorylation in the absence of the test agent indicates that the test agent is an ALK ligand. In certain embodiments, the ALK-expressing cell is a neuroblastoma cell. In specific embodiments, a 5% increase in STAT3 phosphorylation in the presence of the test agent and heparin in comparison with the level of STAT3 phosphorylation in the absence of the test agent indicates that the test agent is an ALK ligand. In specific embodiments, a 10% increase in STAT3 phosphorylation in the presence of the test agent and heparin in comparison with the level of STAT3 phosphorylation in the absence of the test agent indicates that the test agent is an ALK ligand. In specific embodiments, a 25% increase in STAT3 phosphorylation in the presence of the test agent and heparin in comparison with the level of STAT3 phosphorylation in the absence of the test agent indicates that the test agent is an ALK ligand. In specific embodiments, a 50% increase in STAT3 phosphorylation in the presence of the test agent and heparin in comparison with the level of STAT3 phosphorylation in the absence of the test agent indicates that the test agent is an ALK ligand. In specific embodiments, a 5%, 10%, 15%, 25%, 50%, 75%, or 100% increase in STAT3 phosphorylation in the presence of the test agent and heparin in comparison with the level of STAT3 phosphorylation in the absence of the test agent indicates that the test agent is an ALK ligand.

In certain embodiments, provided herein is a method of screening for an ALK ligand, comprising i) contacting an ALK-expressing cell with a test agent and heparin, and ii) measuring the level of STAT5 phosphorylation, wherein an increase in STAT5 phosphorylation in the presence of the test agent and heparin in comparison with the level of STAT5 phosphorylation in the absence of the test agent indicates that the test agent is an ALK ligand. In certain embodiments, the ALK-expressing cell is a neuroblastoma cell. In specific embodiments, a 5% increase in STAT5 phosphorylation in the presence of the test agent and heparin in comparison with the level of STAT5 phosphorylation in the absence of the test agent indicates that the test agent is an ALK ligand. In specific embodiments, a 10% increase in STAT5 phosphorylation in the presence of the test agent and heparin in comparison with the level of STAT5 phosphorylation in the absence of the test agent indicates that the test agent is an ALK ligand. In specific embodiments, a 25% increase in STAT5 phosphorylation in the presence of the test agent and heparin in comparison with the level of STAT5 phosphorylation in the absence of the test agent indicates that the test agent is an ALK ligand. In specific embodiments, a 50% increase in STAT5 phosphorylation in the presence of the test agent and heparin in comparison with the level of STAT5 phosphorylation in the absence of the test agent indicates that the test agent is an ALK ligand. In specific embodiments, a 5%, 10%, 15%, 25%, 50%, 75%, or 100% increase in STAT5 phosphorylation in the presence of the test agent and heparin in comparison with the level of STAT5 phosphorylation in the absence of the test agent indicates that the test agent is an ALK ligand.

In certain embodiments, provided herein is a method of screening for an ALK ligand, comprising i) contacting a neuronal cell with a test agent and heparin, and ii) measuring the level of neurite outgrowth; wherein an increase in neurite outgrowth in the presence of the test agent and heparin in comparison with the level of neurite outgrowth in the absence of the test agent indicates that the test agent is an ALK ligand. In specific embodiments, a 5% increase in neurite outgrowth in the presence of the test agent and heparin in comparison with the level of neurite outgrowth in the absence of the test agent indicates that the test agent is an ALK ligand. In specific embodiments, a 10% increase in neurite outgrowth in the presence of the test agent and heparin in comparison with the level of neurite outgrowth in the absence of the test agent indicates that the test agent is an ALK ligand. In specific embodiments, a 25% increase in neurite outgrowth in the presence of the test agent and heparin in comparison with the level of neurite outgrowth in the absence of the test agent indicates that the test agent is an ALK ligand. In specific embodiments, a 50% increase in neurite outgrowth in the presence of the test agent and heparin in comparison with the level of neurite outgrowth in the absence of the test agent indicates that the test agent is an ALK ligand. In specific embodiments, a 5%, 10%, 15%, 25%, 50%, 75%, or 100% increase in neurite outgrowth in the presence of the test agent and heparin in comparison with the level of neurite outgrowth in the absence of the test agent indicates that the test agent is an ALK ligand.

In certain embodiments, provided herein is a method of screening for an ALK ligand, comprising i) combining the test agent with heparin and ALK, and ii) measuring the level of dimerization of ALK; wherein an increase in dimerization of ALK in the presence of the test agent and heparin in comparison with the level of dimerization of ALK in the absence of the test agent indicates that the test agent is an ALK ligand. In specific embodiments, a 5% increase in dimerization of ALK in the presence of the test agent and heparin in comparison with the level of dimerization of ALK in the absence of the test agent indicates that the test agent is an ALK ligand. In specific embodiments, a 10% increase in dimerization of ALK in the presence of the test agent and heparin in comparison with the level of dimerization of ALK in the absence of the test agent indicates that the test agent is an ALK ligand. In specific embodiments, a 25% increase in dimerization of ALK in the presence of the test agent and heparin in comparison with the level of dimerization of ALK in the absence of the test agent indicates that the test agent is an ALK ligand. In specific embodiments, a 50% increase in dimerization of ALK in the presence of the test agent and heparin in comparison with the level of dimerization of ALK in the absence of the test agent indicates that the test agent is an ALK ligand. In specific embodiments, a 5%, 10%, 15%, 25%, 50%, 75%, or 100% increase in dimerization of ALK in the presence of the test agent and heparin in comparison with the level of dimerization of ALK in the absence of the test agent indicates that the test agent is an ALK ligand.

In certain embodiments, provided herein is a method of screening for an ALK ligand, comprising i) combining the test agent with heparin and the N-terminal domain of ALK, and ii) measuring the level of dimerization of the N-terminal domain of ALK; wherein an increase in dimerization of the N-terminal domain of ALK in the presence of the test agent and heparin in comparison with the level of dimerization of the N-terminal domain of ALK in the absence of the test agent indicates that the test agent is an ALK ligand. In certain embodiments, the N-terminal domain of ALK comprises amino acid 48 to amino acid 65 of human ALK. In certain embodiments, the N-terminal domain of ALK comprises amino acid 21 to amino acid 263 of human ALK. In certain embodiments, the level of dimerization of the N-terminal domain of ALK is measured by size exclusion chromatography combined with multiangle laser light scattering (SEC-MALLS). In specific embodiments, a 5% increase in dimerization of the N-terminal domain of ALK in the presence of the test agent and heparin in comparison with the level of dimerization of the N-terminal domain of ALK in the absence of the test agent indicates that the test agent is an ALK ligand. In specific embodiments, a 10% increase in dimerization of the N-terminal domain of ALK in the presence of the test agent and heparin in comparison with the level of dimerization of ALK in the absence of the test agent indicates that the test agent is an ALK ligand. In specific embodiments, a 25% increase in dimerization of the N-terminal domain of ALK in the presence of the test agent and heparin in comparison with the level of dimerization of the N-terminal domain of ALK in the absence of the test agent indicates that the test agent is an ALK ligand. In specific embodiments, a 50% increase in dimerization of the N-terminal domain of ALK in the presence of the test agent and heparin in comparison with the level of dimerization of the N-terminal domain of ALK in the absence of the test agent indicates that the test agent is an ALK ligand. In specific embodiments, a 5%, 10%, 15%, 25%, 50%, 75%, or 100% increase in dimerization of the N-terminal domain of ALK in the presence of the test agent and heparin in comparison with the level of dimerization of the N-terminal domain of ALK in the absence of the test agent indicates that the test agent is an ALK ligand.

In certain embodiments, provided herein is a method of screening for an ALK ligand, comprising i) contacting the ALK with a test agent and heparin and ii) measuring the level of binding of the test agent to ALK, wherein binding of the test agent to the ALK indicates that the test agent is an ALK ligand. In certain embodiments, binding of the test agent to ALK is not competitive with heparin binding. In certain embodiments, binding of the test agent to ALK is competitive with heparin binding.

In certain embodiments, provided herein is a method of screening for an ALK ligand, comprising i) contacting the N-terminal domain of ALK with a test agent and heparin and ii) measuring the level of binding of the test agent to the N-terminal domain of ALK, wherein binding of the test agent to the N-terminal domain of ALK indicates that the test agent is an ALK ligand. In certain embodiments, the N-terminal domain of ALK comprises amino acid 48 to amino acid 65 of human ALK. In certain embodiments, the N-terminal domain of ALK comprises amino acid 21 to amino acid 263 of human ALK. In certain embodiments, binding of the test agent to the N-terminal domain of ALK is not competitive with heparin binding. In certain embodiments, binding of the test agent to the N-terminal domain of ALK is competitive with heparin binding. In certain embodiments, binding to the N-terminal domain of ALK is measured by isothermal titration calorimetry (ITC). In certain embodiments, binding to the N-terminal domain of ALK is measured by surface plasmon resonance (SPR).

In certain embodiments, provided herein is a method of screening for an ALK ligand, comprising i) contacting ALK with a test agent and heparin, and ii) measuring the level of complex formation between ALK, heparin, and the test agent; wherein formation of a complex between ALK, heparin, and the test agent indicates that the test agent is an ALK ligand. In certain embodiments, complex formation is measured by surface plasmon resonance (SPR).

### 6. EXAMPLES

The examples in this section (i.e., section 6) are offered by way of illustration, and not by way of limitation.

### 6.1 Example 1: The N-Terminal Domain of ALK Contains A Heparin-Binding Motif

An alignment of the amino acid sequences of human ALK and dog ALK with the heparin binding motif found in the FGF receptor (FGFR) family of RTKs revealed that the N-terminal domain of the human and dog ALK also contain putative heparin binding motifs. As shown in FIG. 1, a region within human and dog ALK amino acid residues 44-69 align with the FGFR family heparin binding motifs. Positively charged amino acid residues (depicted in FIG. 1 with a black background) are responsible for binding of FGFR to negatively charged heparin. These residues are largely conserved in ALK along with the intervening sequence, which constitutes the heparin-binding motif. The dotted lines correspond to the approximate location of the motif within the N-terminal domain of the ALK ECD (ALK ECD not drawn to scale).

### 6.2 Example 2: Heparin, but Neither Pleotrophin or Midkine, Induces ALK Phosphorylation

NB1 neuroblastoma cells were treated with heparin (Hep), pleiotrophin (PTN), and midkine (MK) to determine the influence of each on ALK phosphorylation. A Western blot analysis using antibodies specific for phosphorylated ALK and phosphorylated MAPK showed that treating cells with 10 μg/mL Hep stimulates ALK phosphorylation and promotes MAPK activation, as seen in FIG. 2. By contrast, neither PTN nor MK influences ALK phosphorylation. An agonistic anti-ALK monoclonal antibody was used as a positive control for activation of ALK and MAPK. Conversely, an inhibitory anti-ALK antibody specifically blocks heparin-induced activation of ALK and MAPK, demonstrating that activation of ALK by heparin is likely to be direct. Heparin stimulation of NB1 cells also induced ALK internalization (data not shown), similar to ligand-mediated internalization observed with other receptor tyrosine kinases.

### 6.3 Example 3: Dose Dependent Activation of ALK by Heparin and Effect of Treatment of ALK with Various Other Glycosaminoglycans

NB1 neuroblastoma cells were treated with increasing doses of heparin (0, 0.001, 0.01, 0.1, 1, 10, 100 μg/mL heparin), and phosphorylation of ALK and AKT was monitored by Western blot analysis. Various glycosaminoglycans at 10 μg/mL were also assessed for their ability to activate ALK. An ALK-agonistic mAb was used as a positive control for pALK and pAKT. As shown in FIG. 3, treatment with heparin resulted in dose-dependent phosphorylation of ALK and AKT, with the effects of heparin-induced phosphorylation peaking at 10 μg/mL. Dextran sulfate was also shown to induce phosphorylation of ALK and AKT at 10 μg/mL.

### 6.4 Example 4: Heparin Induced Activation of ALK is Inhibited by SOS

Sucrose-octasulfate (SOS) is a heparin mimetic which contains the sugar and sulfate moieties to tightly bind heparin binding sites but lacks the length/size to induce dimerization (classically used to demonstrate the role of heparin in activating FGF receptors). NB1 neuroblastoma cells were treated with increasing doses of SOS (0, 0.001, 0.01, 0.1, 1, and 10 mg/mL) along with 10 μg/mL of heparin. As shown in FIG. 4 SOS is capable of inhibiting heparin-induced phosphorylation of ALK, AKT, and MAPK in a dose dependent manner.

### 6.5 Example 5: ALK Activation is Dependent on Heparin Chain Length

An enzyme-linked immunosorbent assay (ELISA) was used to determine the effect of heparin chain length on its ability to induced ALK phosphorylation. Heparin of specific chain lengths of 2, 4, 9, 12, 15, 20, and 25 were compared with heterogeneous chain-length heparin. NB1 cells were titrated with each heparin variant for 10 minutes, and ALK phosphorylation was read out by ELISA. The graph in FIG. 5 shows that heparin of 15 disaccharides (dp) or greater was able to induce ALK activation. The parabolic nature of ALK activation likely reflects monovalent saturation of ALK binding sites when heparin is in large excess.

### 6.6 Example 6: ALK Activation is Dependent on Heparin Sulfation Pattern

ELISA was used to test the effect of removal and addition of O-sulfated and N-sulfated esters in the heparin chain on ALK phosphorylation. As shown in FIG. 6, removal of O-sulfated or N-sulfated esters in the heparin chain resulted in a loss of ALK phosphorylation. By contrast, heparin oversulfation (whereby most available hydroxyl groups are substituted by O-sulfate esters) promoted more robust ALK phosphorylation.

### 6.7 Example 7: SPR Analysis of Heparin Binding to the ALK FL-ECD and ΔN-ECD

Surface plasmon resonance (SPR) was used to analyze the binding of heparin to the ALK FL-ECD and ΔN-ECD (see Materials and Methods, below) as well as the SOS inhibition of heparin binding to FL-ECD.

FIG. 7 shows the binding affinities of the ALK ECD and NTD deletion mutant to heparin. Using a steady-state model of binding affinity, the FL-ECD bound heparin with a K_{D} of 151 nM, whereas the ΔN-ECD mutant bound to heparin only extremely weakly. The traces shown in FIG. 7 are reference subtracted with a background binding to the reference cell of <5%. Likewise, FL-ECD was capable of being purified using heparin sepharose chromatography, while, in contrast, ΔN-ECD was not (data not shown).

An ALK antibody that was shown to bind to the N-terminal region of ALK (tested via immunoblot, not shown) was able to disrupt the interaction of FL-ECD with heparin in SPR experiments (data not shown).

FIG. 8 shows the SPR analysis wherein a titration of SOS was pre-incubated with 350 nM of FL-ECD. These complexes were then injected over the heparin surface. Rₘₐₓ values were taken for each concentration of SOS and an IC₅₀ was calculated to be 6.5 μM. The inhibition constant (Kᵢ) for SOS was determined to be 2.25 μM. Thus, the results of this experiment demonstrate that SOS inhibits binding of FL-ECD. In addition, the IC₅₀ and inhibition constant values indicate a high degree of specificity for the interaction between heparin and ALK.

Materials and Methods.

Cloning.

The nucleotide sequence coding for amino acids 1-1137 of dog ALK was synthesized by BLUEHERON™. A His-tag was added to the 3′ end followed by a stop codon. An XhoI site was added upstream of the start codon and an XbaI site was added directly after the stop codon. The construct was then subcloned into pCDNA3.3 (Life Technologies).

Expression.

293-S cells cultured were cultured in DMEM-F12 with 5% FBS and 1% penicillin/streptomycin prior to transfection. Culture medium was switched to OPTI-MEM™ just prior to transfection. The cells were then transiently transfected using standard LIPOFECTAMINE 2000™ protocol (Invitrogen). Cells were incubated for 4 days. Media was collected and clarified by centrifugation and vacuum filtration (0.45 PVDF filter).

Purification.

Nickel-sepharose EXCEL™ (GE-Healthcare) beads were added to the clarified media and incubated overnight at 4° C. with agitation. Beads were washed with 20 column volumes (CV) with PBS and 20 CV of 25 mM imidizole, 25 mM Hepes, 150 mM NaCl, 10% Glycerol, pH 7.4. Elution was performed with 500 mM imidizole, 25 mM Hepes, 150 mM NaCl, 10% Glycerol, pH 7.4. Fractions were collected and immediately subjected to SEC via FPLC (size exclusion chromatography: Superdex200 HiLoad. Buffer 25 mM Hepes, 150 mM NaCl, 10% Glycerol, pH 7.4). Fractions containing the desired protein were collected, combined and concentrated to 10 mg/mL using 30,000 MWCO centrifuge device (Sartorius Stedim). Overall yield was ˜4 mg/L of culture with ˜97% purity.

SPR.

A BIACORE™ T100 instrument was used at 25° C. using 25 mM Hepes, 300 mM NaCl, 10% Glycerol, pH 7.4. All reagents were dialyzed with this buffer prior to use. Heparin-biotin (Sigma) sold as ≥97% pure was further purified using PD-10 pre-packed columns (GE-Healthcare) to remove free biotin in solution. Heparin-biotin was then immobilized on an assembled NeutrAvidin surface (amine coupled on a CM4 Series-S Biacore chip): three surfaces were produced with different concentrations of immobilized heparin by varying contact time from 48 to 240 s, and excess ligand was removed. Due to the heterogeneous nature of heparins, the surface was a combination of many chain lengths of heparin. Three-fold dilutions of FL-ECD and ΔN-ECD were injected sequentially over a reference surface without heparin and three heparin surfaces. The surface was regenerated between cycles with 2.5 M NaCl, 5 mM Acetic Acid, pH 4.5. For the SOS competition assay, serial dilutions of SOS were pre-incubated with 0.350 μM FL-ECD and then injected over the chip. The surface was regenerated between cycles with 2.5 M NaCl, 5 mM Acetic Acid, pH 4.5. Data were analyzed using the BiaCore T100 software.

### 6.8 Example 8: ITC Analysis

ITC assays were used to find the stoichiometry and affinity of different chain lengths of heparin for ALK FL-ECD. ITC assays were performed using a VP ITC (Microcal) with a 1.3 mL cell volume and 250 μL ligand syringe volume at 25° C. The buffer used in all cases was 25 mM Hepes, 150 mM NaCl, 10% Glycerol, pH 7.4. Each macromolecule and ligand was extensively dialyzed against this buffer. For dp25 heparin binding to ALK: 1.43 mL of 8.3 μM ALK was placed in the cell. 250 μL of 44 μM dp25 heparin was titrated in 8 μL increments. For dp15 heparin binding to ALK: 1.43 mL of 6 μM ALK was placed in the cell. 250 μL of 60 μM dp25 heparin was titrated in 8 μL increments. For dp9 heparin binding to ALK: 1.43 mL of 10 μM ALK was placed in the cell. 250 μL of 150 μM dp25 heparin was titrated in 10 μL increments. Data were collected, and then processed and analyzed using Origin 5.0 with Microcal ITC feature software. Data were corrected for heat of dilution. Data were then fit to a one-site model by nonlinear least squares regression to calculate affinities and stoichiometries.

TABLE 1
Molar ratio and affinity of ALK:Heparin binding as measured
by isothermal titration calorimetry (ITC)

| Heparin | | | |
|---|---|---|---|
| Length | Molar Ratio | K_{D} | deltaH |
| dp9 | 1 Heparin:0.91 ALK | 0.505 μM | −7.7 kcal/mole |
| dp15 | 1 Heparin:2.3 ALK | 0.200 μM | −18 kcal/mole |
| dp25 | 1 Heparin:4.7 ALK | 0.080 μM | −30 kcal/mole |

The results of the ITC experiments shown in Table 1 and FIG. 9A-C demonstrate that heparin oligomerizes FL-ECD, confirming the high affinity binding of heparin to ALK determined by SPR. Both the stoichiometry and affinity of the ALK-heparin complex increased as a function of the length of the heparin chain, which is indicative of an avidity effect. These biophysical studies are in good agreement with the cell-based data.

### 6.9 Example 9: SEC-MALLS Analysis

An SEC-MALLS analysis was performed to analyze the effect of heparin of different chain lengths on ALK oligomerization. The light scattering data were collected using a tandem of Superose 6 and Superdex 75, 10/300, HR Size Exclusion Chromatography (SEC) column (GE Healthcare, Piscataway, N.J.), connected to High Performance Liquid Chromatography System (HPLC), Agilent 1200, (Agilent Technologies, Wilmington, Del.) equipped with an autosampler. The elution from SEC was monitored by a photodiode array (PDA) UV/VIS detector (Agilent Technologies, Wilmington, Del.), differential refractometer (OPTI-Lab rEx Wyatt Corp., Santa Barbara, Calif.), static and dynamic, multiangle laser light scattering (LS) detector (HELEOS II with QELS capability, Wyatt Corp., Santa Barbara, Calif.). The SEC-UV/LS/RI system was equilibrated in buffer 150 mM NaCl, 25 mM Hepes (pH 7.4), 10% glycerol at the flow rate of 0.4 ml/min. Two software packages were used for data collection and analysis: the Chemstation software (Agilent Technologies, Wilmington, Del.) controlled the HPLC operation and data collection from the multi-wavelength UV/VIS detector, while the ASTRA software (Wyatt Corp., Santa Barbara, Calif.) collected data from the refractive index detector, the light scattering detectors, and recorded the UV trace at 280 nm sent from the PDA detector. The weight average molecular masses, Mw, were determined across the entire elution profile in the intervals of 1 sec from static LS measurement using ASTRA software as previously described (Folta-Stogniew and Williams, 1999).

FIG. 10 shows the results of FL-ECD mixed with dp15, dp25 or buffer and subjected to SEC-MALLS. FL-ECD+dp15 formed a FL-ECD dimer, FL-ALK+dp25 formed a FL-ECD tetramer/pentamer and FL-ECD+buffer formed a FL-ECD monomer. In FIG. 10, the UV traces are thin lines, MALLS traces are thick lines. The results of this experiment demonstrate that increasing the chain length of heparin increases the oligomeric state of ALK.

In contrast, in the presence of heparins with an average chain length of <dp10, FL-ECD eluted from the column as a monomer (data not shown). ΔN-ECD, lacking the heparin binding motif, does not elute at different molecular weights in the presence of dp25 (data not shown), supporting a role for heparin binding in dimerization. The correlation of heparin chain length with dimerization and activation suggests that heparins of certain chain lengths promote dimerization of ALK, resulting in activation of the receptor.

### EQUIVALENTS

The present invention is not to be limited in scope by the specific embodiments described herein. Indeed, various modifications of the invention in addition to those described herein will become apparent to those skilled in the art from the foregoing description. Such modifications are intended to fall within the scope of the appended claims.

All references cited herein are incorporated herein by reference in their entirety and for all purposes to the same extent as if each individual publication, patent or patent application was specifically and individually indicated to be incorporated by reference in its entirety for all purposes.

The citation of any publication is for its disclosure prior to the filing date and should not be construed as an admission that the present invention is not entitled to antedate such publication by virtue of prior invention.

1. A method of treating or preventing a neurodegenerative, neuromuscular or cognitive disease or disorder in a subject, comprising administering to a subject in need thereof a therapeutically effective amount of an anaplastic lymphoma kinase (ALK) activator.

2. (canceled)

3. The method of claim 1, wherein 1.0 μM of the ALK activator is capable of increasing ALK tyrosine phosphorylation above background ALK tyrosine phosphorylation in unstimulated neuroblastoma cells.

4. The method of claim 1, wherein the ALK activator is capable of binding to the N-terminal domain of the ALK receptor.

5. The method of claim 1, wherein the ALK activator is an antibody.

6. The method of claim 1, wherein the ALK activator is capable of binding to a heparin binding motif of the ALK receptor.

7.-11. (canceled)

12. The method of claim 1, wherein the neurodegenerative disease or disorder is Alzheimer's disease.

13.-45. (canceled)

46. A pharmaceutical composition suitable for intraventricular administration comprising an ALK activator.

47.-92. (canceled)

93. A method of treating or preventing an ALK receptor-associated disorder in a subject, comprising administering to a subject in need thereof a therapeutically effective amount of an ALK inhibitor, wherein the ALK inhibitor inhibits binding of heparin or a heparin/chondroitin sulfated growth factor to the ALK receptor.

94. The method of claim 93, wherein the ALK inhibitor inhibits binding of heparin to the N-terminal domain of the ALK receptor.

95. The method of claim 94, wherein the ALK inhibitor inhibits binding of heparin to the heparin binding motif of the ALK receptor.

96.-99. (canceled)

100. The method of claim 93, wherein the ALK inhibitor binds to the ALK receptor.

101. The method of claim 93, wherein the ALK inhibitor is an antibody that specifically binds the ALK receptor.

102. The method of claim 93, wherein the ALK inhibitor is a soluble protein comprising a heparin-binding motif.

103. The method of claim 102, wherein the soluble protein comprises a heparin-binding portion of an ALK N-terminal domain.

104.-105. (canceled)

106. The method of claim 93, wherein the ALK receptor-associated disorder is a hyperproliferative disorder.

107. The method of claim 106, wherein the hyperproliferative disorder is cancer.

108. The method of claim 107, wherein the cancer is a lymphoma.

109. The method of claim 108, wherein the lymphoma is an anaplastic large-cell lymphoma.

110. The method of claim 107, wherein the cancer is a non-small cell lung cancer, inflammatory breast cancer, medulloblastoma, rhabdomyosarcoma, colorectal cancer, pancreatic cancer, myofibroblastic tumors, Ewing's sarcomas, head-and-neck cancer, neurofibromatosis, ovarian cancer, or glioblastoma.

111. The method of claim 107, wherein the cancer is a neuroblastoma.

112.-132. (canceled)


SEQUENCE LISTING (1 sequence)

SEQ ID NO: 1 - protein, 1620 aa, Homo sapiens
  ref <308>: DATABASE ACCESSION NUMBER: GenBank NP_004295.2
  ref <309>: DATABASE ENTRY DATE: 2015-03-15
  ref <313>: RELEVANT RESIDUES IN SEQ ID NO: (1)..(1620)
MGAIGLLWLLPLLLSTAAVGSGMGTGQRAGSPAAGPPLQPREPLSYSRLQRKSLAVDFVVPSLFRVYARDLLLPPSSSELKAGRPEARGSLALDCAPLLRLLGPAPGVSWTAGSPAPAEARTLSRVLKGGSVRKLRRAKQLVLELGEEAILEGCVGPPGEAAVGLLQFNLSELFSWWIRQGEGRLRIRLMPEKKASEVGREGRLSAAIRASQPRLLFQIFGTGHSSLESPTNMPSPSPDYFTWNLTWIMKDSFPFLSHRSRYGLECSFDFPCELEYSPPLHDLRNQSWSWRRIPSEEASQMDLLDGPGAERSKEMPRGSFLLLNTSADSKHTILSPWMRSSSEHCTLAVSVHRHLQPSGRYIAQLLPHNEAAREILLMPTPGKHGWTVLQGRIGRPDNPFRVALEYISSGNRSLSAVDFFALKNCSEGTSPGSKMALQSSFTCWNGTVLQLGQACDFHQDCAQGEDESQMCRKLPVGFYCNFEDGFCGWTQGTLSPHTPQWQVRTLKDARFQDHQDHALLLSTTDVPASESATVTSATFPAPIKSSPCELRMSWLIRGVLRGNVSLVLVENKTGKEQGRMVWHVAAYEGLSLWQWMVLPLLDVSDRFWLQMVAWWGQGSRAIVAFDNISISLDCYLTISGEDKILQNTAPKSRNLFERNPNKELKPGENSPRQTPIFDPTVHWLFTTCGASGPHGPTQAQCNNAYQNSNLSVEVGSEGPLKGIQIWKVPATDTYSISGYGAAGGKGGKNTMMRSHGVSVLGIFNLEKDDMLYILVGQQGEDACPSTNQLIQKVCIGENNVIEEEIRVNRSVHEWAGGGGGGGGATYVFKMKDGVPVPLIIAAGGGGRAYGAKTDTFHPERLENNSSVLGLNGNSGAAGGGGGWNDNTSLLWAGKSLQEGATGGHSCPQAMKKWGWETRGGFGGGGGGCSSGGGGGGYIGGNAASNNDPEMDGEDGVSFISPLGILYTPALKVMEGHGEVNIKHYLNCSHCEVDECHMDPESHKVICFCDHGTVLAEDGVSCIVSPTPEPHLPLSLILSVVTSALVAALVLAFSGIMIVYRRKHQELQAMQMELQSPEYKLSKLRTSTIMTDYNPNYCFAGKTSSISDLKEVPRKNITLIRGLGHGAFGEVYEGQVSGMPNDPSPLQVAVKTLPEVCSEQDELDFLMEALIISKFNHQNIVRCIGVSLQSLPRFILLELMAGGDLKSFLRETRPRPSQPSSLAMLDLLHVARDIACGCQYLEENHFIHRDIAARNCLLTCPGPGRVAKIGDFGMARDIYRASYYRKGGCAMLPVKWMPPEAFMEGIFTSKTDTWSFGVLLWEIFSLGYMPYPSKSNQEVLEFVTSGGRMDPPKNCPGPVYRIMTQCWQHQPEDRPNFAIILERIEYCTQDPDVINTALPIEYGPLVEEEEKVPVRPKDPEGVPPLLVSQQAKREEERSPAAPPPLPTTSSGKAAKKPTAAEISVRVPRGPAVEGGHVNMAFSQSNPPSELHKVHGSRNKPTSLWNPTYGSWFTEKPTKKNNPIAKKEPHDRGNLGLEGSCTVPPNVATGRLPGASLLLEPSSLTANMKEVPLFRLRHFPCGNVNYGYQQQGLPLEAATAPGAGHYEDTILKSKNSMNQPGP